SEQUENCE LISTING (29 sequences)

SEQ ID NO: 1 - DNA, 1494 bp, Artificial sequence
  NA sequence of WT Gcase (E.coli optimized codons)
gcgcgcccgtgcattccgaaaagctttggctatagcagcgtggtgtgcgtgtgcaacgcgacctattgcgatagctttgatccgccgacctttccggcgctgggcacctttagccgctatgaaagcacccgcagcggccgccgcatggaactgagcatgggcccgattcaggcgaaccataccggcaccggcctgctgctgaccctgcagccggaacagaaatttcagaaagtgaaaggctttggcggcgcgatgaccgatgcggcggcgctgaacattctggcgctgagcccgccggcgcagaacctgctgctgaaaagctattttagcgaagaaggcattggctataacattattcgcgtgccgatggcgagctgcgattttagcattcgcacctatacctatgcggataccccggatgattttcagctgcataactttagcctgccggaagaagataccaaactgaaaattccgctgattcatcgcgcgctgcagctggcgcagcgcccggtgagcctgctggcgagcccgtggaccagcccgacctggctgaaaaccaacggcgcggtgaacggcaaaggcagcctgaaaggccagccgggcgatatttatcatcagacctgggcgcgctattttgtgaaatttctggatgcgtatgcggaacataaactgcagttttgggcggtgaccgcggaaaacgaaccgagcgcgggcctgctgagcggctatccgtttcagtgcctgggctttaccccggaacatcagcgcgattttattgcgcgcgatctgggcccgaccctggcgaacagcacccatcataacgtgcgcctgctgatgctggatgatcagcgcctgctgctgccgcattgggcgaaagtggtgctgaccgatccggaagcggcgaaatatgtgcatggcattgcggtgcattggtatctggattttctggcgccggcgaaagcgaccctgggcgaaacccatcgcctgtttccgaacaccatgctgtttgcgagcgaagcgtgcgtgggcagcaaattttgggaacagagcgtgcgcctgggcagctgggatcgcggcatgcagtatagccatagcattattaccaacctgctgtatcatgtggtgggctggaccgattggaacctggcgctgaacccggaaggcggcccgaactgggtgcgcaactttgtggatagcccgattattgtggatattaccaaagataccttttataaacagccgatgttttatcatctgggccattttagcaaatttattccggaaggcagccagcgcgtgggcctggtggcgagccagaaaaacgatctggatgcggtggcgctgatgcatccggatggcagcgcggtggtggtggtgctgaaccgcagcagcaaagatgtgccgctgaccattaaagatccggcggtgggctttctggaaaccattagcccgggctatagcattcatacctatctgtggcatcgccagtaa

SEQ ID NO: 2 - protein, 497 aa, Artificial sequence [sequence omitted]

SEQ ID NO: 3 - DNA, 1494 bp, Artificial sequence
  genetically modified betta-glucocerebrosidase (GCase variant)
gcgcgcccgtgcattccgaaaagctttggctatagcagcgtggtgtgcgtgtgcaacgcgacctattgcgatagctttgatccgccgacctttccggcgctgggcacctttagccgctatgaaagcacccgcagcggccgccgcatggaactgagcatgggcccgattcaggcgaaccataccggcaccggcctgctgctgaccctgcagccggaacagaaatttcagaaagtgaaaggctttggcggcgcgatgaccgatgcggcggcgctgaacattctggcgctgagcccgccggcgcagaacctgctgctgaaaagctattttagcgaagaaggcattggctataacattattcgcgtgccgatggcgagctgcgattttagcattcgcacctatacctatgcggataccccggatgattttcagctgcataactttagcctgccggaagaagataccaaactgaaaattccgctgattcatcgcgcgctgcagctggcgcagcgcccggtgagcctgctggcgagcccgtggaccagcccgacctggctgaaaaccaacggcgcggtgaacggcaaaggcagcctgaaaggccagccgggcgatatttatcatcagacctgggcgcgctattttgtgaaatttctggatgcgtatgcggaacataaactgcagttttgggcggtgaccgcggaaaacgaaccgagcgcgggcctgctgagcggctatccgtttcagtgcctgggctttaccccggaacatcagcgcgattttattgcgcgcgatctgggcccgaccctggcgaacagcacccatcataacgtgcgcctgctgatgctggatgatcagcgcctgctgctgccgcattgggcgaaagtggtgctgaccgatccggaagcggcgaaatatgtgcatggcattgcggtgcattggtatctggattttctggcgccggcgaaagcgaccctgggcgaaacccatcgcctgtttccgaacaccatgctgtttgcgagcgaagcgtgcgtgggcagcaaattttgggaacagagcgtgcgcctgggcagctgggatcgcggcatgcagtatagccatagcattattgaagatctgctgtatcatgtggtgggctggaccgattggaacctggcgctgaacccggaaggcggcccgaactgggtgcgcaactttgtggatagcccgattattgtggatattaccaaagataccttttataaacagccgatgttttatcatctgggccattttagcaaatttattccggaaggcagccagcgcgtgggcctggtggcgagccagaaaaacgatctggatgcggtggcgctgatgcatccggatggcagcgcggtggtggtggtgctgaaccgcagcagcaaagatgtgccgctgaccattaaagatccggcggtgggctttctggaaaccattagcccgggctatagcattcatacctatctgtggcatcgccagtaa

SEQ ID NO: 4 - protein, 497 aa, Artificial sequence
  genetically modified betta-glucocerebrosidase (GCase variant)
ARPCIPKSFGYSSVVCVCNATYCDSFDPPTFPAPGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHNLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIIEDLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWHRQ

SEQ ID NO: 5 - DNA, 1494 bp, Artificial sequence
  genetically modified betta-glucocerebrosidase (GCase variant)
gcgcgcccgtgcattccgaaaagctttggctatagcagcgtggtgtgcgtgtgcaacgcgacctattgcgatagctttgatccgccgacctttccggcgccgggcacctttagccgctatgaaagcacccgcagcggccgccgcatggaactgagcatgggcccgattcaggcgaaccataccggcaccggcctgctgctgaccctgcagccggaacagaaatttcagaaagtgaaaggctttggcggcgcgatgaccgatgcggcggcgctgaacattctggcgctgagcccgccggcgcagaacctgctgctgaaaagctattttagcgaagaaggcattggctataacattattcgcgtgccgatggcgagctgcgattttagcattcgcacctatacctatgcggataccccggatgattttcagctgcataactttagcctgccggaagaagataccaaactgaaaattccgctgattcatcgcgcgctgcagctggcgcagcgcccggtgagcctgctggcgagcccgtggaccagcccgacctggctgaaaaccaacggcgcggtgaacggcaaaggcagcctgaaaggccagccgggcgatatttatcatcagacctgggcgcgctattttgtgaaatttctggatgcgtatgcggaacataacctgcagttttgggcggtgaccgcggaaaacgaaccgagcgcgggcctgctgagcggctatccgtttcagtgcctgggctttaccccggaacatcagcgcgattttattgcgcgcgatctgggcccgaccctggcgaacagcacccatcataacgtgcgcctgctgatgctggatgatcagcgcctgctgctgccgcattgggcgaaagtggtgctgaccgatccggaagcggcgaaatatgtgcatggcattgcggtgcattggtatctggattttctggcgccggcgaaagcgaccctgggcgaaacccatcgcctgtttccgaacaccatgctgtttgcgagcgaagcgtgcgtgggcagcaaattttgggaacagagcgtgcgcctgggcagctgggatcgcggcatgcagtatagccatagcattattgaagatctgctgtatcatgtggtgggctggaccgattggaacctggcgctgaacccggaaggcggcccgaactgggtgcgcaactttgtggatagcccgattattgtggatattaccaaagataccttttataaacagccgatgttttatcatctgggccattttagcaaatttattccggaaggcagccagcgcgtgggcctggtggcgagccagaaaaacgatctggatgcggtggcgctgatgcatccggatggcagcgcggtggtggtggtgctgaaccgcagcagcaaagatgtgccgctgaccattaaagatccggcggtgggctttctggaaaccattagcccgggctatagcattcatacctatctgtggcatcgccagtaa

SEQ ID NO: 6 - protein, 497 aa, Artificial sequence
  genetically modified betta-glucocerebrosidase (GCase variant)
ARPCIPKSFGYSSVVCVCNATYCDSFDPPTFPAPGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLKNFSLPEEDTKLKIPLIHRALQLAQRPVSLLASPWTSPTWLKTNGAVNGKGSLKGQPGDKYHQTWARYFVKFLDAYAKHNLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNFMLFASEACVGSKFWEQSVRLGSWDRGMQYSHSIIEDLNYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWHRQ

SEQ ID NO: 7 - DNA, 1494 bp, Artificial sequence
  genetically modified betta-glucocerebrosidase (GCase variant)
gcgcgcccgtgcattccgaaaagctttggctatagcagcgtggtgtgcgtgtgcaacgcgacctattgcgatagctttgatccgccgacctttccggcgccgggcacctttagccgctatgaaagcacccgcagcggccgccgcatggaactgagcatgggcccgattcaggcgaaccataccggcaccggcctgctgctgaccctgcagccggaacagaaatttcagaaagtgaaaggctttggcggcgcgatgaccgatgcggcggcgctgaacattctggcgctgagcccgccggcgcaggatctgctgctgaaaagctattttagcgaagaaggcattggctataacattattcgcgtgccgatggcgagctgcgattttagcattcgcacctatacctatgcggataccccggatgattttcagctgaaaaactttagcctgccggaagaagataccaaactgaaaattccgctgattcatcgcgcgcagcagctggcgcagcgcccggtgagcctgctggcgagcccgtggaccagcccgacctggctgaaaaccaacggcgcggtgaacggcaaaggcagcctgaaaggccagccgggcgataaatatcatcagacctgggcgcgctattttgtgaaatttctggatgcgtatgcgaaacataacctgaccttttgggcggtgaccgcggaaaacgaaccgagcgcgggcctgattccgggctatccgtttcagtgcctgggctttaccccggaacatcagcgcgattttattgcgcgcgatctgggcccgaccctggcgaacagcacccatcataacgtgcgcctgctgatgctggatgatcagcgcctgctgctgccgcattgggcgaaagtggtgctgaccgatccggaagcggcgaaatatgtgcatggcattgcggtgcattggtatctggattttctggcgccggcgaaagcgaccctgggcgaaacccatcgcctgtttccgaactatatgctgtttgcgagcgaagcgtgcgtgggcagcaaattttgggaacagagcgtgcgcctgggcagctgggatcgcggcatgcagtatagccatagcattattgaagatctgaactatcatgtggtgggctggaccgattggaacctggcgctgaacccggaaggcggcccgaactgggtgcgcaactttgtggatagcccgattattgtggatattaccaaagataccttttataaacagccgatgttttatcatctgggccattttagcaaatttattccggaaggcagccagcgcgtgggcctggtggcgagccagaaaaacgatctggatgcggtggcgctgatgcatccggatggcagcgcggtggtggtggtgctgaaccgcagcagcaaagatgtgccgctgaccatttgggatccggcggtgggctttctggaaaccattagcccgggctatagcattcatacctatctgtggcgccgccagtaa

SEQ ID NO: 8 - protein, 497 aa, Artificial sequence
  genetically modified betta-glucocerebrosidase (GCase variant)
ARPCIPKSFGYSSVVCVCNATYCDSFDPPTFPAPGTFSRYESTRSGRRMELSMGPIQANHTGTGLLLTLQPEQKFQKVKGFGGAMTDAAALNILALSPPAQDLLLKSYFSEEGIGYNIIRVPMASCDFSIRTYTYADTPDDFQLKNFSLPEEDTKLKIPLIHRAQQLAQRPVSLLASPWTSPTWLKTNGAVNGKGSLKGQPGDKYHQTWARYFVKFLDAYAKHNLTFWAVTAENEPSAGLIPGYPFQCLGFTPEHQRDFIARDLGPTLANSTHHNVRLLMLDDQRLLLPHWAKVVLTDPEAAKYVHGIAVHWYLDFLAPAKATLGETHRLFPNYMLFASEACVGSKFWEQSVRLGSWDRGMQYSHSIIEDLNYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIWDPAVGFLETISPGYSIHTYLWRRQ

SEQ ID NO: 9 - DNA, 1494 bp, Artificial sequence
  genetically modified betta-glucocerebrosidase (GCase variant)
gcgcgcccgtgcattccgaaaagctttggctatagcagcgtggtgtgcgtgtgcaacgcgacctattgcgatagctttgatccgccgacctttccggcgccgggcacctttagccgctatgaaagcacccgcagcggccgccgcatggaacgcagcatgggcccgattcaggcgaaccataccggcaccggcctgctgctgaccctgcagccggaacagaaatttcagaaagtgaaaggctttggcggcgcgatgaccgatgcggcggcgctgaacattctggcgctgagcccgccggcgcaggatctgctgctgaaaagctattttagcgaagaaggcattggctataacattattcgcgtgccgatggcgagctgcgattttagcacccgcacctatacctatgcggataccccggatgattttaaactgaaaaactttagcctgccggaagaagataccaaactgaaaattccgctgattcatcgcgcgcagcagctgagccagcgcccggtgagcctgctggcgagcccgtggaccagcccgacctggctgaaaaccaacggcgcggtgaacggcaaaggcagcctgaaaggccagccgggcgataaatatcatcagacctgggcgaactattttgtgaaatttctggatgcgtatgcgaaacataacctgaccttttgggcggtgaccgcggaaaacgaaccgagcgcgggcctgattccgggctatccgtttcagtgcctgggctttaccccggaacatcagcgcgattttattgcgcgcaacctgggcccgaccctggcgaacagcacccatcataacgtgcgcctgctgatgctggatgatcagcgcctgctgctgccgcattgggcgaaagtggtgctgaccgatccggaagcggcgcgctatgtgcatggcattgcggtgcattggtatctggattttctggcgccggcgaaagcgaccctgggcgaaacccatcgcctgtttccgaactatatgctgtttgcgagcgaagcgtgcgtgggcagcaaattttgggaacagagcgtgcgcctgggcagctgggatcgcggcatgcagtatagccatagcattattgaagatctgaactatcatgtggtgggctggaccgattggaacctggcgctgaacccggaaggcggcccgaactgggtgcgcaactttgtggatagcccgattattgtggatattaccaaagataccttttataaacagccgatgttttatcatctgggccattttagcaaatttattccggaaggcagccagcgcgtgggcctggtggcgagccagaaaaacgatctggatgcggtggcgctgatgcatccggatggcagcgcggtggtggtggtgctgaaccgcagcagcaaagatgtgccgctgaccatttgggatccggcggtgggctttctggaaaccattagcccgggctatagcattcatacctatctgtggcgccgccagtaa

SEQ ID NO: 10 - protein, 497 aa, Artificial sequence
  genetically modified betta-glucocerebrosidase (GCase variant)
ARPCIPKSFGYSSVVCVCNATYCDSFDPPTFPAPGTFSRYESTRSGRRMELSMGPIQANHTGTGLLLTLQPEQKFQKVKGFGGAMTDAAALNILALSPPAQDLLLKSYFSEEGIGYNIIRVPMASCDFSTRTYTYADTPDDFQLKNFSLPEEDTKLKIPLIHRAQQLSQRPVSLLASPWTSPTWLKTNGAVNGKGSLKGQPGDKYHQTWARYFVKFLDAYAKHNLTFWAVTAENEPSAGLIPGYPFQCLGFTPEHQRDFIARNLGPTLANSTHHNVRLLMLDDQRLLLPHWAKVVLTDPEAAKYVHGIAVHWYLDFLAPAKATLGETHRLFPNYMLFASEACVGSKFWEQSVRLGSWDRGMQYSHSIIEDLNYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIWDPAVGFLETISPGYSIHTYLWRRQ

SEQ ID NO: 11 - DNA, 1494 bp, Artificial sequence
  genetically modified betta-glucocerebrosidase (GCase variant)
gcgcgcccgtgcattccgaaaagctttggctatagcagcgtggtgtgcgtgtgcaacgcgacctattgcgatagctttgatccgccgacctttccggcgccgggcacctttagccgctatgaaagcacccgcagcggccgccgcatggaactgagcatgggcccgattcaggcgaaccataccggcaccggcctgctgctgaccctgcagccggaacagaaatttcagaaagtgaaaggctttggcggcgcgatgaccgatgcggcggcgctgaacattctggcgctgagcccgccggcgcaggatctgctgctgaaaagctattttagcgaagaaggcattggctataacattattcgcgtgccgatggcgagctgcgattttagcacccgcacctatacctatgcggataccccggatgattttcagctgaaaaactttagcctgccggaagaagataccaaactgaaaattccgctgattcatcgcgcgcagcagctgagccagcgcccggtgagcctgctggcgagcccgtggaccagcccgacctggctgaaaaccaacggcgcggtgaacggcaaaggcagcctgaaaggccagccgggcgataaatatcatcagacctgggcgaactattttgtgaaatttctggatgcgtatgcgaaacataacctgaccttttgggcggtgaccgcggaaaacgaaccgagcgcgggcctgattccgggctatccgtttcagtgcctgggctttaccccggaacatcagcgcgattttattgcgcgcaacctgggcccgaccctggcgaacagcacccatcataacgtgcgcctgctgatgctggatgatcagcgcctgctgctgccgcattgggcgaaagtggtgctgaccgatccggaagcggcgcgctatgtgcatggcattgcggtgcattggtatctggattttctggcgccggcgaaagcgaccctgggcgaaacccatcgcctgtttccgaactatatgctgtttgcgagcgaagcgtgcgtgggcagcaaattttgggaacagagcgtgcgcctgggcagctgggatcgcggcatgcagtatagccatagcattattgaagatctgaactatcatgtggtgggctggaccgattggaacctggcgctgaacccggaaggcggcccgaactgggtgcgcaactttgtggatagcccgattattgtggatattaccaaagataccttttataaacagccgatgttttatcatctgggccattttagcaaatttattccggaaggcagccagcgcgtgggcctggtggcgagccagaaaaacgatctggatgcggtggcgctgatgcatccggatggcagcgcggtggtggtggtgctgaaccgcagcagcaaagatgtgccgctgaccatttgggatccggcggtgggctttctggaaaccattagcccgggctatagcattcatacctatctgtggcgccgccagtaa

SEQ ID NO: 12 - protein, 497 aa, Artificial sequence
  genetically modified betta-glucocerebrosidase (GCase variant)
ARPCIPKSFGYSSVVCVCNATYCDSFDPPTFPAPGTFSRYESTRSGRRMELSMGPIQANHTGTGLLLTLQPEQKFQKVKGFGGAMTDAAALNILALSPPAQDLLLKSYFSEEGIGYNIIRVPMASCDFSTRTYTYADTPDDFQLKNFSLPEEDTKLKIPLIHRAQQLSQRPVSLLASPWTSPTWLKTNGAVNGKGSLKGQPGDKYHQTWANYFVKFLDAYAKHNLTFWAVTAENEPSAGLIPGYPFQCLGFTPEHQRDFIARNLGPTLANSTHHNVRLLMLDDQRLLLPHWAKVVLTDPEAARYVHGIAVHWYLDFLAPAKATLGETHRLFPNYMLFASEACVGSKFWEQSVRLGSWDRGMQYSHSIIEDLNYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIWDPAVGFLETISPGYSIHTYLWRRQ

SEQ ID NO: 13 - DNA, 1494 bp, Artificial sequence
  NA sequence of optimized ecoli D7 Gcase
gcgcgcccgtgcattccgaaaagctttggctatagcagcgtggtgtgcgtgtgcaacgcgacctattgcgatagctttgatccgccgacctttccggcgccgggcacctttagccgctatgaaagcacccgcagcggccgccgcatggaactgagcatgggcccgattcaggcgaactggaccggcaccggcctgctgctgaccctgcagccggaacagaaatttcagaaagtgaaaggctttggcggcgcgatgaccgatgcggcggcgctgaacattctggcgctgagcccgccggcgcaggataacctgctgaaaagctattttagcgaagaaggcattggctataacattattcgcgtgccgatggcgagctgcgattttagcacccgcacctatacctatgcggataccccggatgattttcagctgaaaaactttagcctgccggaagaagataccaaactgaaaattccgctgattcatcgcgcgcaggcgctgagccagcgcccggtgagcctgctggcgagcccgtggaccagcccgacctggctgaaaaccaacggcgcggtgaacggcaaaggcagcctgaaaggccagccgggcgataaatatcatcagacctgggcgaactattttgtgaaatttctggatgcgtatgcgaaacataacctgaccttttgggcggtgaccgcggaaaacgaaccgagcgcgggcctgattccgggctatccgtttcagtgcctgggctttaccccggaacatcagcgcgattttattgcgcgcaacctgggcccgaccctggcgaacagcacccatcgcaacgtgcgcctgctgatgctggatgatcagcgcctgctgctgccgcattgggcgaaagtggtgctgaccgatccggaagcggcgcgctatgtgcatggcattgcggtgcattggtatctggattttctggcgccggcgaaagcgaccctgggcgaaacccatcgcctgtttccggattatatgctgtttgcgagcgaagcgtgcgtgggcagcaaattttgggaacagagcgtgcgcctgggcagctgggatcgcggcatgcagtatagccatagcattattgaagatctgaactatcatgtggtgggctggaccgattggaacctggcgctggatccggaaggcggcccgaactgggtgaaaaactttgtggatagcccgattattgtggataccaccaaagataccttttataaacagccgatgttttatcatatgggccattttagcaaatttattccggaaggcagccagcgcgtgggcctggtggcgagccagaaaaacgatctggatgcggtggcgctgatgcatccggatggcagcgcggtggtggtggtgctgaaccgcagcagcaaagatgtgccgctgaccatttgggatccggcggtgggctttctggaaaccattagcccgggctatagcattcatacctatctgtggcgccgccagtaa

SEQ ID NO: 14 - protein, 497 aa, Artificial sequence
  AA sequence of D7 Gcase
ARPCIPKSFGYSSVVCVCNATYCDSFDPPTFPAPGTFSRYESTRSGRRMELSMGPIQANWTGTGLLLTLQPEQKFQKVKGFGGAMTDAAALNILALSPPAQDNLLKSYFSEEGIGYNIIRVPMASCDFSTRTYTYADTPDDFQLKNFSLPEEDTKLKIPLIHRAQALSQRPVSLLASPWTSPTWLKTNGAVNGKGSLKGQPGDKYHQTWANYFVKFLDAYAKHNLTFWAVTAENEPSAGLIPGYPFQCLGFTPEHQRDFIARNLGPTLANSTHRNVRLLMLDDQRLLLPHWAKVVLTDPEAARYVHGIAVHWYLDFLAPAKATLGETHRLFPDYMLFASEACVGSKFWEQSVRLGSWDRGMQYSHSIIEDLNYHVVGWTDWNLALDPEGGPNWVKNFVDSPIIVDTTKDTFYKQPMFYHMGHFSKFIPEGSQRVGLVASQKNDLDAVALMHPDGSAVVVVLNRSSKDVPLTIWDPAVGFLETISPGYSIHTYLWRRQ

SEQ ID NO: 15 - DNA, 24 bp, Artificial sequence
  NA sequence of FLAG peptide
gactacaaagacgatgacgacaag

SEQ ID NO: 16 - protein, 8 aa, Artificial sequence
  AA sequence of FLAG peptide
DYKDDDDK

SEQ ID NO: 17 - DNA, 1494 bp, Artificial sequence
  genetically modified betta-glucocerebrosidase (GCase variant)
gcccgcccctgcatccctaaaagcttcggctacagctcggtggtgtgtgtctgcaatgccacatactgtgactcctttgaccccccgacctttcctgcccccggtaccttcagccgctatgagagtacacgcagtgggcgacggatggagctgagtatggggcccatccaggctaattggacgggcacaggcctgctactgaccctgcagccagaacagaagttccagaaaatcaagggatttggaggggccatgacagatgctgctgctctcaacatccttaagctgtcaccccctgcccaagagaacctacttaaatcgtacttctctgaagaaggaatcggatataacatcatccgggtacccatggccagctgtgacttctccacccgcacctacacctatgcagacacccctgatgatttccagttgaagaacttcagcctcccagaggaagataccaagctcaagatacccctgattcaccgagcccaggccttgagccagcgtcccgtttcactccttgccagcccctggacatcacccacttggctcaagaccaatggagcgatgaatgggaaggggtcactcaagggacagcccggagacaagtaccaccagacctgggccaactactttgtgaagttcctggatgcctatgctaagcacggcttaaccttctgggcagtgacagctgaaaatgagccttctgctgggctgatccccggataccccttccagtgcctgggcttcacccctgaacatcagcgagacttcattgcccgtaacctaggtcctaccctcgccaacagtactcaccacaatgtccgcctactcatgctggatgaccaacgcttgctgctgccccactgggcaaaggtggtactgacagacccagaagcagctaaatatgttcatggcattgctgtacattggtacctggactttctggctccagccaaagacaccctaggggagacacaccgcctgttccccaacaagatgctctttgcctcagaggcctgtaccggctccaagttctgggagcagagtgtgcggctaggctcctgggatcgaggggagcagtacgcccacagcatcatcgaggacctcaactactgggtggtcggctggaccgactggaaccttgccctgaaccccgaaggaggacccaattgggtgcgtaactttgtcgacagtcccatcattgtagacgccaccaaggacgagttttacaaacagcccatgttctaccacatcggccacttcagcaagttcattcctgagggctcccagagagtggggctggttgccagtcagaagaacgacctggacgcagtggcactgatgaaccccgatggctctgctgttgtggtcgtgctaaaccgctcctctaaggatgtgcctcttaccatctgggatcctgctgtgggcttcatcgagacaatctcacctggctactccattcacacctacctgtggcgtcgccagtga

SEQ ID NO: 18 - protein, 497 aa, Artificial sequence
  genetically modified betta-glucocerebrosidase (GCase variant)
ARPCIPKSFGYSSVVCVCNATYCDSFDPPTFPAPGTFSRYESTRSGRRMELSMGPIQANWTGTGLLLTLQPEQKFQKIKGFGGAMTDAAALNILKLSPPAQENLLKSYFSEEGIGYNIIRVPMASCDFSTRTYTYADTPDDFQLKNFSLPEEDTKLKIPLIHRAQALSQRPVSLLASPWTSPTWLKTNGAMNGKGSLKGQPGDKYHQTWANYFVKFLDAYAKHGLTFWAVTAENEPSAGLIPGYPFQCLGFTPEHQRDFIARNLGPTLANSTHHNVRLLMLDDQRLLLPHWAKVVLTDPEAAKYVHGIAVHWYLDFLAPAKDTLGETHRLFPNKMLFASEACTGSKFWEQSVRLGSWDRGEQYAHSIIEDLNYWVVGWTDWNLALNPEGGPNWVRNFVDSPIIVDATKDEFYKQPMFYHIGHFSKFIPEGSQRVGLVASQKNDLDAVALMNPDGSAVVVVLNRSSKDVPLTIWDPAVGFIETISPGYSIHTYLWRRQ

SEQ ID NO: 19 - DNA, 1494 bp, Artificial sequence
  genetically modified betta-glucocerebrosidase (GCase variant)
gcccgcccctgcatccctaaaagcttcggctacagctcggtggtgtgtgtctgcaatgccacatactgtgactcctttgaccccccgacctttcctgcccccggtaccttcagccgctatgagagtacacgcagtgggcgacggatggagctgagtatggggcccatccaggctaattggacgggcacaggcctgctactgaccctgcagccagaacagaagttccagaaaatcaagggatttggaggggccatgacagatgctgctgctctcaacatccttaagctgtcaccccctgcccaagagaacctacttaaatcgtacttctctgaagaaggaatcggatataacatcatccgggtacccatggccagctgtgacttctccacccgcacctacacctatgcagacacccctgatgatttccagttgaagaacttcagcctcccagaggaagataccaagctcaagatacccctgattaagcgagcccaggccttgagccagcgtcccgtttcactccttgccagcccctggacagcccccacttggctcaagaccaatggagcgatgaatgggaaggggtcactcaagggacagcccggagacaagtaccaccagacctgggccaactactttgtgaagttcctggatgcctatgctaagcacggcttaaccttctgggcagtgacagctgaaaatgagccttctgctgggctgatccccggataccccttccagtgcctgggcttcacccctgaacatcagcgagacttcattgcccgtaacctaggtcctaccctcgccaacagtactcaccacaatgtccgcctactcatgctggatgaccaacgcttgctgctgccccactgggcaaaggtggtactgagcgacccagaagcagctaaatatgttcatggcattgctgtacattggtacctggactttctggctccagccaaagacaccctaggggagacacaccgcctgttccccaacaagttcctctttgcctcagaggcctgtaccggctcccacttctgggagcagagtgtgcggctaggctcctgggatcgaggggagcagtacgcccacagcatcatcgaggacctcaactactgggtggtcggctggaccgactggaaccttgccctggaccccgaaggaggacccaattgggtgaagaactttgtcgacagtcccatcattgtagacgccaccaaggacgagttttacaaacagcccatgttctaccacatcggccacttcagcaagttcattcctgagggcgcccagagagtggggctggttgccagtcagaagaacgacctggacgcagtggcactgatgaaccccgatggctctgctgttgtggtcgtgctaaaccgctccgacaaggatgtgcctcttaccatctgggatcctgacgtgggcttcatcgagacaatctcacctggctactccattcacacctacctgtggcgtcgccagtga

SEQ ID NO: 20 - protein, 497 aa, Artificial sequence
  genetically modified betta-glucocerebrosidase (GCase variant)
ARPCIPKSFGYSSVVCVCNATYCDSFDPPTFPAPGTFSRYESTRSGRRMELSMGPIQANWTGTGLLLTLQPEQKFQKIKGFGGAMTDAAALNILKLSPPAQENLLKSYFSEEGIGYNIIRVPMASCDFSTRTYTYADTPDDFQLKNFSLPEEDTKLKIPLIKRAQALSQRPVSLLASPWTAPTWLKTNGAMNGKGSLKGQPGDKYHQTWANYFVKFLDAYAKHGLTFWAVTAENEPSAGLIPGYPFQCLGFTPEHQRDFIARNLGPTLANSTHHNVRLLMLDDQRLLLPHWAKVVLSDPEAAKYVHGIAVHWYLDFLAPAKDTLGETHRLFPNKFLFASEACTGSHFWEQSVRLGSWDRGEQYAHSIIEDLNYWVVGWTDWNLALDPEGGPNWVKNFVDSPIIVDATKDEFYKQPMFYHIGHFSKFIPEGAQRVGLVASQKNDLDAVALMNPDGSAVVVVLNRSDKDVPLTIWDPDVGFIETISPGYSIHTYLWRRQ

SEQ ID NO: 21 - DNA, 1494 bp, Artificial sequence
  genetically modified betta-glucocerebrosidase (GCase variant)
gcccgcccctgcatccctaaaagcttcggctacagctcggtggtgtgtgtctgcaatgccacatactgtgactcctttgaccccccgacctttcctgcccccggtaccttcagccgctatgagagtacacgcagtgggaagcggatggagaggagtatggggcccatccaggctaattggacgggcacaggcctgctactgaccctgcacccagaacagaagttccagaaaatcaagggatttggaggggccatgacagatgctgctgctatcaacatccttaagctgtcaccccctgcccaagaggagctacttaaatcgtacttctctgaagaaggaatcgagtataacatcatccgggtacccatgggcagctgtgacttctccacccgcacctacacctatgcagacacccctaacgatttccagttgaagaacttcagcctcccagaggaagataccaagctcaagatacccctgattaagcgagcccaggccttgagccagcgtcccgtttcactccttgccagcccctggacagcccccacttggctcaagaccaatggagcgatgaatgggaaggggaccctcaagggacagcccggagacaagtaccaccagacctgggccaactactttgtgaagttcctggatgcctatgctaagcacggcttaaccttctgggcagtgacagctgaaaatgagccttctgctgggctgatccccggataccccttccagtgcctgggcttcacccctgaacatcagcgagacttcattgcccgtaacctaggtcctaccctcgccaacagtactcaccacaatgtccgcctactcatgctggatgaccaacgcttgctgctgccccactgggcaaaggtggtactgagcgacccagaagcagctaaatatgttcatggcattgctgtacattggtacctggactttctggctccagccaaagacaccctaggggagacacaccgcctgttccccgacaagttcctctttgcctcagaggcctgtaccggctcccacttctgggagcagagtgtgcggctaggctcctgggatcgaggggagcagtacgcccacagcatcatcgaggacctcaactactgggtggtcggctggaccgactggaaccttgccctggaccccgaaggaggacccaattgggtgaagaactttgtcgacagtcccatcattgtagacgccaccaaggacgagttttacaaacagcccatgttctaccacatgggccacttcagcaagttcattcctgagggcgcccagagagtggggctgagcgccagtcagaagaacgacctggacgcagtggcactgatgaaccccgatggctctgctgttgtggtcgtgctaaaccgctccgacaaggatgtgcctcttaccatctgggatcctgacgtgggcttcatcgagacaatctcacctggctactccattcacacctacctgtggcgtcgccagtga

SEQ ID NO: 22 - protein, 497 aa, Artificial sequence
  genetically modified betta-glucocerebrosidase (GCase variant)
ARPCIPKSFGYSSVVCVCNATYCDSFDPPTFPAPGTFSRYESTRSGKRMERSMGPIQANWTGTGLLLTLHPEQKFQKIKGFGGAMTDAAAINILKLSPPAQEELLKSYFSEEGIEYNIIRVPMGSCDFSTRTYTYADTPNDFQLKNFSLPEEDTKLKIPLIKRAQALSQRPVSLLASPWTAPTWLKTNGAMNGKGTLKGQPGDKYHQTWANYFVKFLDAYAKHGLTFWAVTAENEPSAGLIPGYPFQCLGFTPEHQRDFIARNLGPTLANSTHHNVRLLMLDDQRLLLPHWAKVVLSDPEAAKYVHGIAVHWYLDFLAPAKDTLGETHRLFPDKFLFASEACTGSHFWEQSVRLGSWDRGEQYAHSIIEDLNYWVVGWTDWNLALDPEGGPNWVKNFVDSPIIVDATKDEFYKQPMFYHMGHFSKFIPEGAQRVGLSASQKNDLDAVALMNPDGSAVVVVLNRSDKDVPLTIWDPDVGFIETISPGYSIHTYLWRRQ

SEQ ID NO: 23 - DNA, 1494 bp, Artificial sequence; identical to SEQ ID NO: 21
  NA sequence of optimized human D7 Gcase

SEQ ID NO: 24 - DNA, 1494 bp, Artificial sequence
  NA sequence of WT Gcase (human optimized codons)
gcccgcccctgcatccctaaaagcttcggctacagctcggtggtgtgtgtctgcaatgccacatactgtgactcctttgaccccccgacctttcctgcccttggtaccttcagccgctatgagagtacacgcagtgggcgacggatggagctgagtatggggcccatccaggctaatcacacgggcacaggcctgctactgaccctgcagccagaacagaagttccagaaagtgaagggatttggaggggccatgacagatgctgctgctctcaacatccttgccctgtcaccccctgcccaaaatttgctacttaaatcgtacttctctgaagaaggaatcggatataacatcatccgggtacccatggccagctgtgacttctccatccgcacctacacctatgcagacacccctgatgatttccagttgcacaacttcagcctcccagaggaagataccaagctcaagatacccctgattcaccgagccctgcagttggcccagcgtcccgtttcactccttgccagcccctggacatcacccacttggctcaagaccaatggagcggtgaatgggaaggggtcactcaagggacagcccggagacatctaccaccagacctgggccagatactttgtgaagttcctggatgcctatgctgagcacaagttacagttctgggcagtgacagctgaaaatgagccttctgctgggctgttgagtggataccccttccagtgcctgggcttcacccctgaacatcagcgagacttcattgcccgtgacctaggtcctaccctcgccaacagtactcaccacaatgtccgcctactcatgctggatgaccaacgcttgctgctgccccactgggcaaaggtggtactgacagacccagaagcagctaaatatgttcatggcattgctgtacattggtacctggactttctggctccagccaaagccaccctaggggagacacaccgcctgttccccaacaccatgctctttgcctcagaggcctgtgtgggctccaagttctgggagcagagtgtgcggctaggctcctgggatcgagggatgcagtacagccacagcatcatcacgaacctcctgtaccatgtggtcggctggaccgactggaaccttgccctgaaccccgaaggaggacccaattgggtgcgtaactttgtcgacagtcccatcattgtagacatcaccaaggacacgttttacaaacagcccatgttctaccaccttggccacttcagcaagttcattcctgagggctcccagagagtggggctggttgccagtcagaagaacgacctggacgcagtggcactgatgcatcccgatggctctgctgttgtggtcgtgctaaaccgctcctctaaggatgtgcctcttaccatcaaggatcctgctgtgggcttcctggagacaatctcacctggctactccattcacacctacctgtggcgtcgccagtga

SEQ ID NO: 25 - protein, 497 aa, Artificial sequence
  AA sequence of WT Gcase
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQ

SEQ ID NO: 26 - DNA, 1494 bp, Artificial sequence
  genetically modified betta-glucocerebrosidase (GCase variant)
gcccgcccctgcatccctaaaagcttcggctacagctcggtggtgtgtgtctgcaatgccacatactgtgactcctttgaccccccgacctttcctgcccccggtcagttcgcccgctatgagagtacacgcagtgggaagcggatggagaggagtatggggcccatccaggctaattggacgggcacaggcctgctactgaccctgcacccagaacagaagttccagaaaatcaagggatttggaggggccatgacagatgctgctgctatcaacatccttaagctgtcaccccctgcccaagagaggctacttaaatcgtacttctctgaagaaggaatcgagtataacatcatccgggtacccatgggcagctgtgacttctccacccgcacctacacctatgcagacacccctgacgatttcgagttgagaaacttcagcctcccagaggaagataccaagctcaagatacccctgattaagcgagcccaggccttgagccagcgtcccgtttcactccttgccagcccctggacagcccccgcctggatgaagaccaatggagcgatgaatgggaaggggaccctcaagggacagcccggagacaagtaccaccagacctgggccaactactttgtgaagttcctggatgcctatgctaagcacggcttaaccttctgggcagtgacagctgaaaatgagccttctgctgggctgatccccggataccccttccagtgcctgggcttcacccctgaacatcagcgagacttcattgcccgtaacctaggtcctaccctcgccaacagtagccacaaggacgtccgcctactcatgctggatgaccaacgcagcctgctgccccactgggcacaggtggtactgagcgacccaagagcagctaaatatgttcatggcattgctgtacattggtacctggactttctggctccagccgaggacaccctaggggagacacaccgcctgttccccgacaagttcctctttgcctcagaggcctgtaccggctcccacttctgggagcagagtgtgcggctaggctcctgggatcgaggggagcagtacgcccacagcatcatcgaggacctcaactactgggtgaccggctggaccgactggaaccttgccctggaccccgaaggaggacccaattgggtgaagaactttgtcgacagtcccatcattgtagacgccaccagggacgagttttacaaacagcccatgttctaccacatgggccacttcagcaagttcattcctgagggcgcccagagagtggggctgagcgccagtgagaagaacgacctggacgcagtggcactgcagaaccccgatggctctgctgttgtggtcgtgctaaaccgctccgacaaggatgtgcctcttaccatctgggatcctgacgtgggcttcatcgagacagtgtcacctggctactccattcacacctacctgtggcgtcgccagtga

SEQ ID NO: 27 - protein, 497 aa, Artificial sequence
  genetically modified betta-glucocerebrosidase (GCase variant)
ARPCIPKSFGYSSVVCVCNATYCDSFDPPTFPAPGQFARYESTRSGKRMERSMGPIQANWTGTGLLLTLHPEQKFQKIKGFGGAMTDAAAINILKLSPPAQERLLKSYFSEEGIEYNIIRVPMGSCDFSTRTYTYADTPDDFELRNFSLPEEDTKLKIPLIKRAQALSQRPVSLLASPWTAPAWMKTNGAMNGKGTLKGQPGDKYHQTWANYFVKFLDAYAKHGLTFWAVTAENEPSAGLIPGYPFQCLGFTPEHQRDFIARNLGPTLANSSHKDVRLLMLDDQRSLLPHWAQVVLSDPRAAKYVHGIAVHWYLDFLAPAEDTLGETHRLFPDKFLFASEACTGSHFWEQSVRLGSWDRGEQYAHSIIEDLNYWVTGWTDWNLALDPEGGPNWVKNFVDSPIIVDATRDEFYKQPMFYHMGHFSKFIPEGAQRVGLSASEKNDLDAVALQNPDGSAVVVVLNRSDKDVPLTIWDPDVGFIETVSPGYSIHTYLWRRQ

SEQ ID NO: 28 - DNA, 87 bp, Artificial sequence
  NA TwinStrep
tggtcccacccccagttcgagaagggcggcggtagtggagggggcggatctggcggctcagcttggagccacccccagttcgaaaag

SEQ ID NO: 29 - protein, 29 aa, Artificial sequence
  AA TwinStrep
WSHPQFEKGGGSGGGGSGGSAWSHPQFEK

(19) United States
(12) Patent Application Publication
(10) Pub. No.: US 2023/0132790 A1
(43) Pub. Date: May 4, 2023

(54) VARIANTS OF BETA-GLUCOCEREBROSIDASE FOR USE IN TREATING GAUCHER DISEASE

(71) Applicant: Yeda Research and Development Co. Ltd., Rehovot (IL)

(72) Inventors: Anthony H. FUTERMAN, Rehovot (IL); Israel SILMAN, Rehovot (IL); Joel L. SUSSMAN, Rehovot (IL); Sarel FLEISHMAN, Rehovot (IL); Adi GOLDENZWEIG, Rehovot (IL); Sarka POKORNA, Rehovot (IL); Yaacov ASHANI, Rehovot (IL); Olga KHERSONSKY, Rehovot (IL)

(73) Assignee: Yeda Research and Development Co. Ltd., Rehovot (IL)

(21) Appl. No.: 17/915,500

(22) PCT Filed: Mar. 29, 2021
(86) PCT No.: PCT/IL2021/050357

§ 371 Date: Sep. 29, 2022

Related U.S. Application Data
(60) Provisional application No. 63/049,685, filed on Jul. 9, 2020.

(30) Foreign Application Priority Data
Mar. 29, 2020 (IL) 273684

(51) Int. Cl. C12N 9/24

(52) U.S. Cl. CPC C12N 9/2402; C12Y 302/01045; A61K 38/00

(57) ABSTRACT

A genetically modified human beta-glucocerebrosidase (GCase) is disclosed. The genetically modified GCase comprising an amino acid sequence at least 85% identical to SEQ ID NO: 2; and comprising mutations at coordinates L34P, K224N/G, T369E and N370D, where the coordinates correspond to said SEQ ID NO: 2; and capable of catalyzing hydrolysis of a glycolipid glucosylceramide (GlcCer). Pharmaceutical compositions comprising the genetically modified GCase and therapeutic methods of using same are also disclosed.

### RELATED APPLICATION

This application claims the benefit of priority of Israel Application No. 273684 filed on Mar. 29, 2020 and U.S. Provisional Patent Application No. 63/049,685 filed on Jul. 9, 2020, the contents of which are incorporated herein by reference in their entirety.

### SEQUENCE LISTING STATEMENT

The ASCII file, entitled 86504SequenceListing.txt, created on 29 Mar. 2021, comprising 81,406 bytes, submitted concurrently with the filing of this application is incorporated herein by reference.

### FIELD AND BACKGROUND OF THE INVENTION

The present invention, in some embodiments thereof, relates to variants of β-glucocerebrosidase (GCase), and more particularly, but not exclusively, to the use of same for the treatment of β-glucocerebrosidase deficiency diseases, including Gaucher Disease.

Lysosomal storage disorders (LSDs) encompass about 50 different inherited diseases. They are caused by deficiencies in lysosomal enzymes or transporters, resulting in intra-lysosomal accumulation of undegraded metabolites. Among LSDs, Gaucher Disease (GD) is the most prevalent;

it is caused by mutations in the GBA1 gene. The GBA1 gene encodes beta-glucocerebrosidase (also called acid beta-glucosidase, D-glucosyl-N-acylsphingosine glucohydrolase, or GCase), a lysosomal enzyme with glucosylceramidase activity that is needed to cleave, by hydrolysis, the beta-glucosidic linkage of glucosylceramide (GlcCer, also called glucocerebroside), an intermediate in glycolipid metabolism. As a consequence, cells accumulate large quantities of GlcCer, and eventually die.

From a clinical perspective, GD can be divided into three sub-types based on age of onset and on signs of nervous system involvement. The major symptoms of Type 1 GD, the most common form of the disease, are enlargement of spleen and liver, anemia, thrombocytopenia, and skeletal lesions. Type 2 and 3 GD, the neuropathic forms of GD (nGD), are classified according to the time of onset and rate of progression of neurological symptoms. Type 2, the acute neuropathic form, usually refers to children who display neurological abnormalities before 6 months of age and die by 2-4 years of age. In Type 3, the sub-acute, chronic neuropathic form, patients present with similar symptoms to those observed in Type 2, but with a later onset and severity.

Type 1 GD patients are typically treated by Enzyme Replacement Therapy (ERT). Ceredase®

(Alglucerase), the first drug for GD targeted ERT (a placenta-derived product) was approved by the FDA in 1991, and has been withdrawn from the market due to the approval of similar drugs made by recombinant DNA technology including Imiglucerase (Cerezyme®), approved in 1995; Velaglucerase alpha (VPRIV®), approved in 2010; and Taliglucerase alfa (Elelyso®), approved in 2012. These therapies are not a cure for GD, that is, they do not correct the underlying genetic defect. Thus to benefit from the treatment, symptomatic patients need to continue with ERT for life.

In addition to the aforementioned ERT treatments, Miglustat (OGT 918, N-butyl-deoxynojirimycin) (Zavesca®) a small molecule, orally available drug, approved in 2002, provides substrate reduction therapy (SRT) for the treatment of GD. Zavesca® reduces the harmful buildup of glycosphingolipids (GSLs) throughout the body by reducing the amount of GSLs that the body produces. Additionally, Eliglustat (Cerdelga®), approved in 2014, is also a small molecule used for the treatment of GD. Cerdelga® is believed to work by inhibition of glucosylceramide synthase.

A retrospective analysis of Miglustat for Type 1 GD has found that a combination therapy may offer GD patients better disease control (by employing more than one mechanism of action against the accumulation of glucosylceramide in cells), can be cost-effective, by permitting use of reduced doses of both ERT and Miglustat, and can provide an acceptable quality of life [Machaczka M. et al., Upsala Journal of Medical Sciences (2012) 117, 28-34].

### SUMMARY OF THE INVENTION

According to an aspect of some embodiments of the present invention there is provided a genetically modified human β-glucocerebrosidase (GCase): (i) comprising an amino acid sequence at least 85% identical to SEQ ID NO: 2; and (ii) comprising mutations at coordinates L34P, K224N/G, T369E and N370D, where the coordinates correspond to SEQ ID NO: 2; and (iii) capable of catalyzing hydrolysis of a glycolipid glucosylceramide (GlcCer).

According to an aspect of some embodiments of the present invention there is provided an isolated polynucleotide comprising a nucleic acid sequence encoding the genetically modified human GCase of some embodiments of the invention.

According to an aspect of some embodiments of the present invention there is provided a nucleic acid construct comprising the isolated polynucleotide of some embodiments of the invention, and a cis-acting regulatory element for directing expression of the nucleic acid sequence in a cell.

According to an aspect of some embodiments of the present invention there is provided an isolated cell comprising the polynucleotide of some embodiments of the invention, or construct of some embodiments of the invention.

According to an aspect of some embodiments of the present invention there is provided a pharmaceutical composition comprising as an active ingredient the genetically modified human GCase of some embodiments of the invention, the isolated polynucleotide of some embodiments of the invention, the construct of some embodiments of the invention, or the cell of some embodiments of the invention, and a pharmaceutically acceptable carrier or diluent.

According to an aspect of some embodiments of the present invention there is provided a method of treating a disease associated with β-glucocerebrosidase deficiency in a subject in need thereof, the method comprising administering to the subject a therapeutically effective amount of the genetically modified human GCase of some embodiments of the invention, the isolated polynucleotide of some embodiments of the invention, the construct of some embodiments of the invention, or the cell of some embodiments of the invention, thereby treating the disease associated with the β-glucocerebrosidase deficiency in the subject.

According to an aspect of some embodiments of the present invention there is provided a therapeutically effective amount of the genetically modified human GCase of some embodiments of the invention, the isolated polynucleotide of some embodiments of the invention, the construct of some embodiments of the invention, or the cell of some embodiments of the invention, for use in treating a disease associated with β-glucocerebrosidase deficiency in a subject in need thereof.

According to some embodiments of the invention, the genetically modified human GCase further comprises at least one of the mutations: H145K/R, I204K, E222K, T334F/Y/K and/or L372N, where the coordinates correspond to SEQ ID NO: 2.

According to some embodiments of the invention, the genetically modified human GCase further comprises at least one of the mutations: N102D/E, L165Q, Q226T, L241I, S242P, K473W and/or H495R, where the coordinates correspond to SEQ ID NO: 2.

According to some embodiments of the invention, the genetically modified human GCase further comprises at least one of the mutations: I130T, A168S and/or D263N, where the coordinates correspond to SEQ ID NO: 2.

According to some embodiments of the invention, the genetically modified human GCase further comprises at least one of the mutations: R211N and/or K303R, where the coordinates correspond to SEQ ID NO: 2.

According to some embodiments of the invention, the genetically modified human GCase further comprises at least one of the mutations: H60W, L103N/E/R, Q166A, H274R, N333D, N386D, R395K, I406T/A and/or L420M/I, where the coordinates correspond to SEQ ID NO: 2.

According to some embodiments of the invention, the genetically modified human GCase further comprises at least one of the mutations: V78I, A95K, V191M, A322D, V343T, M361E, S364A, H374W, T410E, H451N and/or L480I, where the coordinates correspond to SEQ ID NO: 2.

According to some embodiments of the invention, the genetically modified human GCase further comprises at least one of the mutations: H162K, S181A, T297S, M335F, K346H, S431A,

S465D and/or A476D, where the coordinates correspond to SEQ ID NO: 2.

According to some embodiments of the invention, the genetically modified human GCase further comprises at least one of the mutations: R47K, L51R, Q70H, L91I, G115E, A124G, D140N/G, S196T, and/or V437S, where the coordinates correspond to SEQ ID NO: 2.

According to some embodiments of the invention, the genetically modified human GCase further comprises at least one of the mutations: T36Q, S38A, Q143E, T183A, L185M, T272S, H274K, N275D, L286S, K293Q, E300R, K321E, V376T, K408R, Q440E, M450Q, and/or I483V, where the coordinates correspond to SEQ ID NO: 2.

According to some embodiments of the invention, the amino acids at coordinates D127, F128, W179, N234, E235, Y244, F246, Q284, Y313, E340, 5345, W381, N396, where the coordinates correspond to SEQ ID NO: 2, are not modified.

According to some embodiments of the invention, the amino acid sequence is identical to a sequence selected from the group consisting of SEQ ID NO: 4, 6, 8, 10, 12, 14, 18, 20, 22 and 27.

According to some embodiments of the invention, the amino acid sequence is as set forth in SEQ ID NO: 14.

According to some embodiments of the invention, the amino acid sequence is as set forth in SEQ ID NO: 22.

According to some embodiments of the invention, the amino acid sequence is as set forth in SEQ ID NO: 27.

According to some embodiments of the invention, the genetically modified human GCase is capable of catalyzing hydrolysis of the artificial substrate p-nitrophenyl-β-D-glucopyranoside (p-NP-Glc).

According to some embodiments of the invention, the genetically modified human GCase is capable of catalyzing hydrolysis of GlcCer by at least about 0.2×10⁶k_{cat}/Kₘ (M⁻¹min⁻¹).

According to some embodiments of the invention, the genetically modified human GCase is capable of catalyzing hydrolysis of GlcCer by at least about 0.5×10⁶k_{cat}/Kₘ (M⁻¹min⁻¹).

According to some embodiments of the invention, the genetically modified human GCase is capable of catalyzing hydrolysis of GlcCer by at least about 1.5×10⁶k_{cat}/Kₘ (M⁻¹min⁻¹).

According to some embodiments of the invention, the genetically modified human GCase comprises a thermal stability under a temperature range being 5-20° C. higher compared to a GCase polypeptide under the same conditions.

According to some embodiments of the invention, the genetically modified human GCase comprises a thermal stability under a temperature range being 5-20° C. higher compared to a wild-type polypeptide under the same conditions.

According to some embodiments of the invention, the genetically modified human GCase comprises a thermal stability under a temperature range being at least 5° C. higher compared to a wild-type GCase polypeptide under the same conditions. According to some embodiments of the invention, the genetically modified human GCase comprises a thermal stability under a temperature range being at least 10° C. higher (e.g. at least 12° C. higher) compared to a wild-type GCase polypeptide under the same conditions.

According to some embodiments of the invention, the genetically modified human GCase comprises a thermal stability under a temperature range being 5-20° C. higher compared to a Cerezyme® polypeptide under the same conditions.

According to some embodiments of the invention, the genetically modified human GCase comprises a thermal stability under a temperature range being at least 10° C. higher (e.g. at least 11° C. higher) compared to a ^{Cerezyme}® polypeptide under the same conditions.

According to some embodiments of the invention, the genetically modified human GCase comprises a thermal stability under a temperature range being at least 15° C. higher (e.g. at least 17° C. higher) compared to a ^{Cerezyme}® polypeptide under the same conditions.

According to some embodiments of the invention, the genetically modified human GCase comprises at least 2 times higher intracellular expression level in eukaryotic cells as compared to a wild-type polypeptide under the same culture conditions.

According to some embodiments of the invention, the genetically modified human GCase is secreted from eukaryotic cells as compared to a wild-type polypeptide not being secreted under the same culture conditions.

According to some embodiments of the invention, the isolated polynucleotide comprises the nucleic acid sequence as set forth in SEQ ID NOs: 3, 5, 7, 9, 11, 13, 17, 19, 21, 23 or 26.

According to some embodiments of the invention, the cis-acting regulatory element comprises a promoter.

According to some embodiments of the invention, the disease associated with β-glucocerebrosidase deficiency is Gaucher disease.

According to some embodiments of the invention, the subject is a human being.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example, and for purposes of illustrative discussion of embodiments of the invention. In this regard, the written description, taken with the drawings, makes apparent to those skilled in the art how embodiments of the invention may be practiced.

In the drawings:

FIG. 1A illustrates the amino acid sequences of wild-type (WT, SEQ ID NO: 2, comprising a single R495H mutation as presented by Cerezyme®, Sanofi Genzyme) and variants D2-D7 GCase (set forth in SEQ ID NOs: 4, 6, 8, 10, 12 and 14, respectively). The active site residues of the enzyme are underlined in each of the sequences. All mutations introduced into the sequences of D2-D7 GCase variants by PROSS are highlighted in bold.

FIG. 1B illustrates the amino acid sequences of wild-type (WT) and variant D7 GCase. The complete amino acid sequence of GCase WT is shown in the upper row (SEQ ID NO: 2), mutations introduced into the D7 sequence by PROSS are shown in the lower row (SEQ ID NO: 14).

FIG. 2A is a schematic representation of the GCase sequence position within the pCDNA3.1 vector used for GCase expression in HEK293T cells.

FIGS. 2B-2C are photographs illustrating sodium dodecyl sulfate/polyacrylamide gel electrophoresis (SDS-PAGE) of the three eluate fractions obtained by purification of GCase isolated from the WT and GCase pellets (FIG. 2B), and SDS-PAGE of secreted GCase purified on FLAG beads (FIG. 2C). Of note, only the D7 variant yielded a secreted enzyme. Arrows indicate the position of the GCase band identified by Mass Spectrometry (MS).

FIGS. 3A-B illustrate size exclusion chromatography (SEC) of D7 GCase after one-step purification using FLAG beads. The protein fractions eluted from the FLAG beads with FLAG peptide were pooled, and applied to a Superdex200 column. Protein was monitored by absorbance at 280 nm. Fractions corresponding to each peak were collected, concentrated, and analyzed by SDS-PAGE. Of note, Peak 1 corresponds to the monomeric GCase.

FIG. 4 is a graph illustrating representative Michaelis-Menten plots for WT GCase (triangles, full line) and D7 GCase (circles, dotted line). The rate of substrate to product conversion (y-axis) is normalized to 1 μg/ml of protein.

FIG. 5A illustrates the amino acid sequences of wild-type (WT, SEQ ID NO: 2) and variants D7, D13, D14 and D15 GCase (set forth in SEQ ID NOs: 14, 18, 20 and 22, respectively). The active site residues of the enzyme are underlined in each of the sequences. All mutations introduced into the sequences of D7, D13, D14 and D15 GCase variants by PROSS are highlighted in bold.

FIG. 5B illustrates a comparison of amino acid sequences of wild type GCase (upper sequence, set forth in SEQ ID NO: 2) and PROSS designed variant D15 GCase (lower sequence, set forth in SEQ ID NO: 22). Mutated amino acids are highlighted in bold, amino acids corresponding to enzymatic catalytic site are underlined.

FIG. 6 illustrates the specific activity of variant D15 GCase (open circles) and Cerezyme® (black circles) estimated using p-NP-Glc as a substrate. Activity was determined substrate concentrations: 0.4, 1.5 and 3 mM p-NP-Glc. FIG. 7 illustrates the amino acid sequences of wild-type (WT, SEQ ID NO: 2, comprising a single R495H mutation as presented by Cerezyme®, Sanofi Genzyme), wild-type (WT, SEQ ID NO: 25) and variants D7, D13, D14, D15 and D16 GCase (set forth in SEQ ID NOs: 14, 18, 20, 22 and 27, respectively). The active site residues of the enzyme are underlined in each of the sequences. All mutations introduced into the sequences of D7, D13, D14, D15 and D16 GCase variants by PROSS are highlighted in bold.

### DESCRIPTION OF SPECIFIC EMBODIMENTS OF THE INVENTION

The present invention, in some embodiments thereof, relates to variants of β-glucocerebrosidase (GCase), and more particularly, but not exclusively, to the use of same for the treatment of β-glucocerebrosidase deficiency diseases, including Gaucher Disease (GD).

The principles and operation of the present invention may be better understood with reference to the drawings and accompanying descriptions.

Before explaining at least one embodiment of the invention in detail, it is to be understood that the invention is not necessarily limited in its application to the details set forth in the following description or exemplified by the Examples. The invention is capable of other embodiments, or of being practiced or carried out in various ways. Also, it is to be understood that the phraseology and terminology employed herein is for the purpose of description, and should not be regarded as limiting.

GD is a genetic lysosomal storage disorder caused by functional deficiency of β-glucocerebrosidase (GCase) that results in multiple organ malfunctions. GCase catalyses the hydrolysis of glucocerebroside to ceramide and glucose. In GD, the enzyme deficiency results in accumulation of excessive glucocerebroside in lysosomal compartments of Gaucher cells (tissue macrophages), and in accumulation of these cells in the visceral tissues (liver, spleen and bone marrow). GD patients are typically treated by Enzyme Replacement Therapy (ERT). Three enzymes are commercially available for the treatment of GD by ERT. These include Imiglucerase (Cerezyme®), .Velaglucerase alpha (VPRIV®) and Taliglucerase alfa (Elelyso®). One drawback associated with current ERT treatments is that the in vivo bioactivity of the enzyme is undesirably low. This is due to, for example, low thermal stability, low uptake, reduced targeting to lysosomes of the specific cells where the substrate accumulates, and/or a short functional in vivo half-life in the lysosomes.

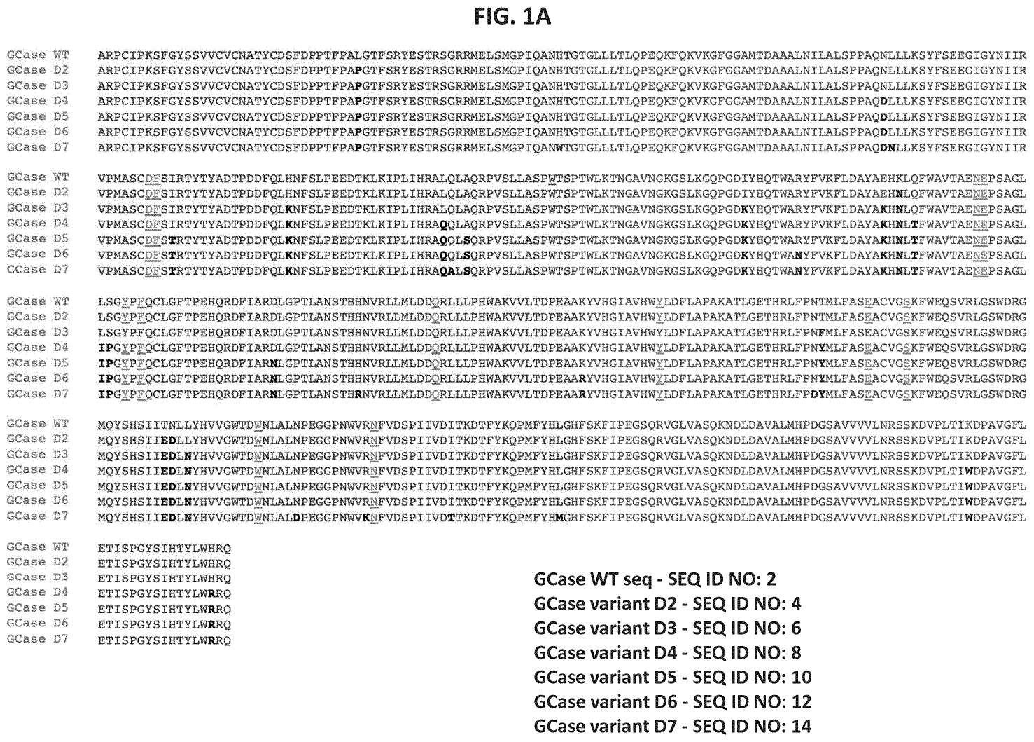

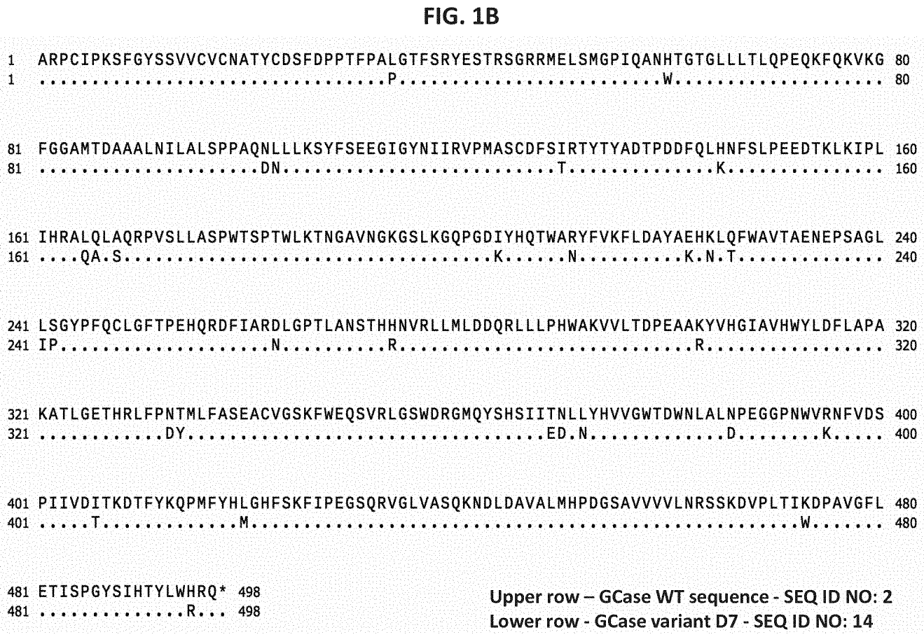

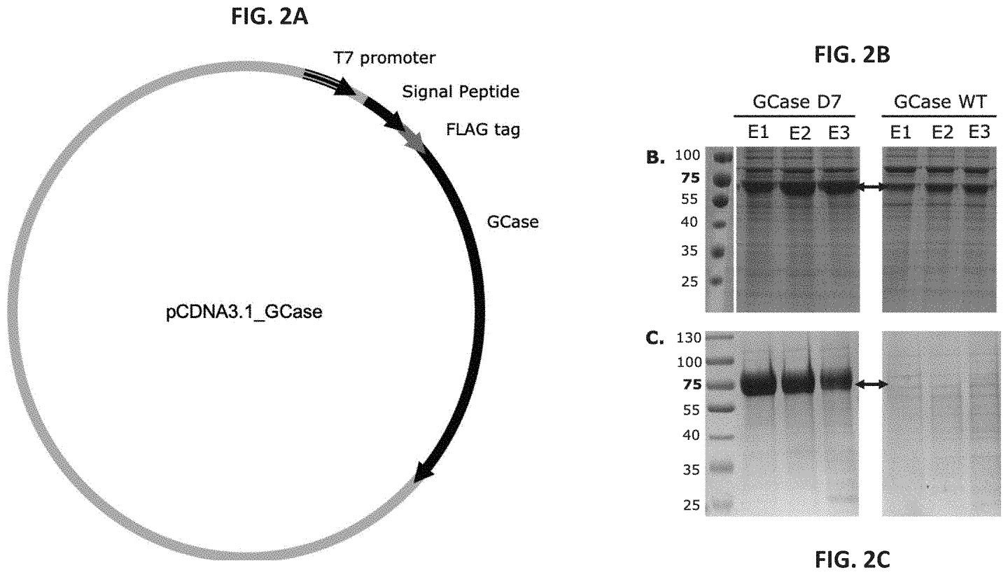

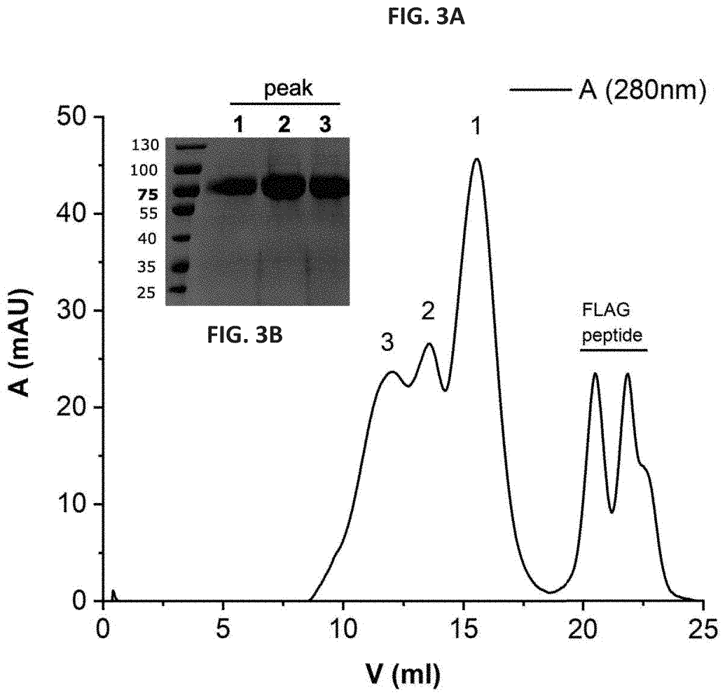

While reducing the present invention to practice, the present inventors have generated new GCases for ERT of Gaucher disease comprising improved properties, i.e., higher expression levels (as compared to wild-type human GCase), higher thermal stability (as compared to wild-type human GCase and/or to Cerezyme®), while maintaining enzymatic activity (as compared to wild-type human GCase). Specifically, the present inventors have generated six new polypeptide variants of GCase by use of the PROSS algorithm, described e.g. in PCT/IL2016/050812 and Goldenzweig A. et al., Mol. Cell. (2016) 63: 337-346, incorporated herein by reference (designs 2-7, i.e. D2-7, set forth in SEQ ID NOs: 4, 6, 8, 10, 12 and 14, respectively, see FIG. 1A). Four of these GCase variants, D2, D4, D6 and D7, were expressed in E. coli and shown to display enzymatic activity towards a synthetic substrate, p-NP-Glc (data not shown). Recombinant human glucosylceramidase (WT human GCase, set forth in SEQ ID NO: 2) and the D7 variant, which bears 30 mutations (set forth in SEQ ID NO: 14), were further expressed in human embryonic kidney cells (HEK293T cells) which are capable of protein glycosylation. As illustrated in FIGS. 2B-C, the D7 GCase showed a higher intracellular expression level as compared to the WT hGCase, while only D7 GCase was secreted from HEK293T cells. With regard to thermal stability, D7 GCase displayed a higher thermal stability by about 11° C. and about 20° C. as compared to Cerezyme® at pH 6.1 and at pH 7.4, respectively, and higher thermal stability by about 7° C. as compared to wild-type human GCase at pH 6.1 (see Table 1, below). With regard to enzymatic activity, D7 GCase and WT hGCase had similar k_{cat}/Kₘ values (see Table 2, below). Taken together, the novel variants of GCase (D2-7 GCases) are endowed with higher expression levels and higher thermal stability, while maintaining enzymatic activity, as compared to wild-type human GCase, and are endowed with higher thermal stability as compared to Cerezyme®. Therefore, they offer promising new possibilities for use in Enzyme Replacement Therapy (ERT) for treatment of GD.

The present inventors have further generated four new polypeptide variants of GCase by use of the PROSS algorithm, (designs 13-16, i.e. D13-16, set forth in SEQ ID NOs: 18, 20, 22 and 27, respectively). GCase variants D13, D14, D15 and D16 were expressed in HEK293T cells, isolated from culture medium and tested for enzymatic activity using fluorescently labelled analogue of GCase (NBD glucosylceramide (d18:1/6:0) (C6NBD GlcCer)). The designs with highest enzymatic activity, i.e. variants D15 and D16 GCase were used for further characterization. As evident from Examples 6 and 9 below, variants D15 and D16 GCase displayed a higher thermal stability by 17-20° C. when compared to Cerezyme® at pH 6.1. Furthermore, enzymatic activity as determined by both natural substrate of GCase (C₆NBD GlcCer) and synthetic substrate (p-NP-Glc), was comparable for Cerezyme® and variants D15 and D16 (see Examples 7 and 10, below).

Thus, according to one aspect of the present invention, there is provided a genetically modified human β-glucocerebrosidase (GCase): (i) comprising an amino acid sequence at least 85% identical to SEQ ID NO: 2; and (ii) comprising mutations at coordinates L34P, K224N/G, T369E and N370D, where the coordinates correspond to SEQ ID NO: 2; and (iii) capable of catalyzing hydrolysis of a glycolipid glucosylceramide (GlcCer).

As used herein, the term “beta-glucocerebrosidase” or “glucocerebrosidase” (EC 3.2.1.45), also referred to as glucosylceramidase, acid beta-glucosidase, D-glucosyl-N-acylsphingosine glucohydrolase, GCase or GBA, refers to an enzyme with glucosylceramidase activity. The β-glucocerebrosidase (GCase) of this aspect of the present invention is a human GCase that catalyzes the hydrolysis of glucosylceramide/GlcCer (an intermediate in glycolipid metabolism) into ceramide and glucose.

According to one embodiment, the protein on which modifications are performed comprises a sequence as set forth SEQ ID NO: 2.

According to one embodiment, the protein on which modifications are performed comprises a sequence as set forth SEQ ID NO: 25 (UniProtKB - P04062 (GLCM_HUMAN)).

It will be appreciated that the sequence as set forth SEQ ID NO: 2 comprises one modification compared to the human wild type GCase at amino acid position 495, i.e. the arginine (R) at position 495 of the human wild type GCase is replaced with histidine (H), to arrive at SEQ ID NO: 2. The enzymatic activity of GCase is not influenzed by this modification.

According to a specific embodiment, the sequence of the human GCase protein is as set forth in SEQ ID NO: 25 (UniProtKB-P04062 (GLCM_HUMAN). The positions of the mutations between SEQ ID NO: 2 and SEQ ID 25 (human) are identical.

As used herein, the term “catalytic domain” of GCase refers to the amino acid residues involved in catalyzing the hydrolysis of glucosylceramide/GlcCer. The 3D structure of the catalytic domain forms the active site, so GCase needs to be correctly folded to be active. For example, the catalytic domain of GCase comprises amino acids coordinates D127, F128, W179, N234, E235, Y244, F246, Q284, Y313, E340, 5345, W381, N396 of SEQ ID NO: 2 or SEQ ID NO: 25 (UniProtKB -P04062 (GLCM_HUMAN).

As used herein, the term “glycosylation site” refers to asparagine residues of GCase to which glycoside chains are attached posttranslationally, and whose presence enhances the activity of the enzyme. In human GCase, there are five candidate sites, N19, N59, N146, N270, and N462. N462 is not typically occupied. It was previously shown by Berg-Fussman and coworkers (Berg-Fussman, Grace, Ionnou & Grabowski [1993] J Biol Chem 268:14861-14866) that if these asparagines are mutated to glutamines, thus preventing glycosylation, GCase activity is significantly, though not completely, reduced. While the glycoside chains differ (in the various recombinant forms expressed), the proximal sugar is an N-aceylglucosamine moiety to which several mannose residues are attached.

According to one embodiment, the genetically modified human GCase (also referred to as variant or polypeptide) comprises an amino acid sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% identical to SEQ ID NO: 2 or to SEQ ID NO: 25.

According to a specific embodiment, the genetically modified human GCase comprises an amino acid sequence at least 84% identical to SEQ ID NO: 2 or to SEQ ID NO: 25.

According to a specific embodiment, the genetically modified human GCase comprises an amino acid sequence at least 85% identical to SEQ ID NO: 2 or to SEQ ID NO: 25.

According to a specific embodiment, the genetically modified human GCase comprises an amino acid sequence at least 86% identical to SEQ ID NO: 2 or to SEQ ID NO: 25.

According to a specific embodiment, the genetically modified human GCase comprises an amino acid sequence at least 88% identical to SEQ ID NO: 2 or to SEQ ID NO: 25.

According to a specific embodiment, the genetically modified human GCase comprises an amino acid sequence at least 90% identical to SEQ ID NO: 2 or to SEQ ID NO: 25.

According to a specific embodiment, the genetically modified human GCase comprises an amino acid sequence at least 95% identical to SEQ ID NO: 2 or to SEQ ID NO: 25.

According to a specific embodiment, the genetically modified human GCase comprises an amino acid sequence at least 96% identical to SEQ ID NO: 2 or to SEQ ID NO: 25.

According to a specific embodiment, the genetically modified human GCase comprises an amino acid sequence at least 97% identical to SEQ ID NO: 2 or to SEQ ID NO: 25.

According to a specific embodiment, the genetically modified human GCase comprises an amino acid sequence at least 98% identical to SEQ ID NO: 2 or to SEQ ID NO: 25.

According to a specific embodiment, the genetically modified human GCase comprises an amino acid sequence at least 99% identical to SEQ ID NO: 2 or to SEQ ID NO: 25.

Homology (e.g., percent homology, sequence identity +sequence similarity) can be determined using any homology comparison software computing a pairwise sequence alignment.

As used herein, “sequence identity” or “identity” in the context of two nucleic acid or polypeptide sequences includes reference to the residues in the two sequences which are the same when aligned. When percentage of sequence identity is used in reference to proteins it is recognized that residue positions which are not identical often differ by conservative amino acid substitutions, where amino acid residues are substituted for other amino acid residues with similar chemical properties (e.g. charge or hydrophobicity), and do not, therefore, change the functional properties of the molecule. Where sequences differ in conservative substitutions, the percent sequence identity may be adjusted upwards to correct for the conservative nature of the substitution. Sequences which differ by such conservative substitutions are considered to have “sequence similarity” or “similarity”. Means for making this adjustment are well-known to those of skill in the art. Typically, this involves scoring a conservative substitution as a partial rather than a full mismatch, thereby increasing the percentage sequence identity. Thus, for example, where an identical amino acid is given a score of 1, and a non-conservative substitution is given a score of zero, a conservative substitution is given a score between zero and 1. The scoring of conservative substitutions is calculated, e.g., according to the algorithm of Henikoff S and Henikoff JG. [Amino acid substitution matrices from protein blocks. Proc. Natl. Acad. Sci. U.S.A. 1992, 89(22): 10915-9].

Identity (e.g., percent homology) can be determined using any homology comparison software, including for example, the BlastN software of the National Center of Biotechnology Information (NCBI) such as by using default parameters.

According to some embodiments of the invention, the identity is a global identity, i.e., an identity over the entire amino acid or nucleic acid sequences of the invention and not over portions thereof.

According to some embodiments of the invention, the term “homology” or “homologous” refers to identity of two or more nucleic acid sequences; or identity of two or more amino acid sequences; or the identity of an amino acid sequence to one or more nucleic acid sequences.

According to some embodiments of the invention, the homology is a global homology, i.e., a homology over the entire amino acid or nucleic acid sequences of the invention and not over portions thereof.

The degree of homology or identity between two or more sequences can be determined using various known sequence comparison tools. Following is a non-limiting description of such tools that can be used along with some embodiments of the invention.

Pairwise global alignment was defined by S. B. Needleman and C. D. Wunsch, “A general method applicable to the search of similarities in the amino acid sequence of two proteins” Journal of Molecular Biology, 1970, pages 443-53, volume 48).

When starting from a polypeptide sequence and comparing to polynucleotide sequences, the OneModel FramePlus algorithm [Halperin, E., Faigler, S. and Gill-More, R. (1999)—FramePlus: aligning DNA to protein sequences. Bioinformatics, 15, 867-873) (available from biocceleration(dot)com/Products(dot)html) can be used.

When starting with a polynucleotide sequence and comparing to other polynucleotide sequences the EMBOSS-6.0.1 Needleman-Wunsch algorithm (available from embos s(dot)sourceforge(dot)net/apps/cvs/emboss/apps/needle(dot)html) can be used.

According to some embodiments, determination of the degree of homology further requires employing the Smith-Waterman algorithm (for protein-protein comparison or nucleotide-nucleotide comparison).

According to some embodiments of the invention, the global homology is performed on sequences which are pre-selected by local homology to the polypeptide or polynucleotide of interest (e.g., 60% identity over 60% of the sequence length), prior to performing the global homology to the polypeptide or polynucleotide of interest (e.g., 80% global homology on the entire sequence). For example, homologous sequences are selected using the BLAST software with the Blastp and tBlastn algorithms as filters for the first stage, and the needle (EMBOSS package) or Frame+ algorithm alignment for the second stage. Local identity (Blast alignments) is defined with a very permissive cutoff—60% Identity on a span of 60% of the sequences lengths because it is used only as a filter for the global alignment stage. In this specific embodiment (when the local identity is used), the default filtering of the Blast package is not utilized (by setting the parameter “-F F”). In the second stage, homologs are defined based on a global identity of at least 80% to the core gene polypeptide sequence.

According to some embodiments of the invention, the GCase polypeptide is 470-520 amino acids in length.

According to some embodiments of the invention, the GCase polypeptide is 480-510 amino acids in length.

According to some embodiments of the invention, the GCase polypeptide is 490-510 amino acids in length.

According to some embodiments of the invention, the GCase polypeptide is 495-500 amino acids in length.

According to a specific embodiment, the GCase polypeptide comprises an amino acid sequence comprising 480, 481, 482, 483, 484, 485, 486, 487, 488, 489, 490, 491, 492, 493, 494, 495, 496, 497, 498, 499, 500, 501, 502, 503, 504 or 505 amino acid residues.

According to a specific embodiment, the GCase polypeptide comprises an amino acid sequence comprising 497 amino acid residues.

The term “polypeptide” as used herein encompasses modifications rendering the polypeptides highly expressible, more stable both in vitro and in vivo, within an animal or human body, or more capable of penetrating into cells as compared to the native GCase sequence i.e., SEQ ID NO: 2 or SEQ ID NO: 25.

Such modifications include, but are not limited to N terminus modification, C terminus modification, polypeptide bond modification, backbone modifications, and residue modification. Methods for preparing peptidomimetic compounds are well known in the art, and are specified, for example, in Quantitative Drug Design, C. A. Ramsden Gd., Chapter 17.2, F. Choplin Pergamon Press (1992), which is incorporated by reference as if fully set forth herein. Further details in this respect are provided herein under.

The term “isolated” refers to at least partially separated from the natural environment e.g., the human body. According to one embodiment, the isolated polypeptide is essentially free from contaminating cellular components, such as carbohydrates, lipids, or other proteinaceous impurities associated with the polypeptide in nature. However, the term “isolated” does not exclude the presence of the same polypeptide in alternative physical forms, such as dimers or alternatively glycosylated or derivatized forms.

The term “amino acid” or “amino acids” is understood to include the 20 naturally occurring amino acids; those amino acids often modified post-translationally in vivo, including, for example, hydroxyproline, phosphoserine and phosphothreonine; and other unusual amino acids including, but not limited to, 2-aminoadipic acid, hydroxylysine, isodesmosine, nor-valine, nor-leucine and ornithine.

According to one embodiment, the amino acid is an “equivalent amino acid residue”. An equivalent amino acid residue refers to an amino acid residue capable of replacing another amino acid residue in a polypeptide without substantially altering the structure and/or functionality of the polypeptide (e.g. capability of catalyzing the hydrolysis of glucosylceramide/GlcCer). Equivalent amino acids thus have similar properties, such as bulkiness of the side-chain, side chain polarity (polar or non-polar), hydrophobicity (hydrophobic or hydrophilic), pH (acidic, neutral or basic) and side chain organization of carbon molecules (aromatic/aliphatic). As such, “equivalent amino acid residues” can be regarded as “conservative amino acid substitutions”.

Within the meaning of the term “equivalent amino acid substitution” one amino acid may be substituted for another within the groups of amino acids indicated herein below:
i) Amino acids having polar side chains (Asp, Glu, Lys, Arg, His, Asn, Gln, Ser, Thr, Tyr, Cys);
ii) Amino acids having non-polar side chains (Gly, Ala, Val, Leu, Ile, Phe, Trp, Pro, Met);
iii) Amino acids having non-polar aliphatic side chains (Gly, Ala, Val, Leu, Ile);
iv) Amino acids having cyclic side chains (Phe, Tyr, Trp, His, Pro);
v) Amino acids having aromatic side chains (Phe, Tyr, Trp);
vi) Amino acids having acidic side chains (Asp, Glu);
vii) Amino acids having basic side chains (Lys, Arg, His);
viii) Amino acids having amide side chains (Asn, Gln);
ix) Amino acids having hydroxy side chains (Ser, Thr);
x) Amino acids having sulphur-containing side chains (Cys, Met);
xi) Neutral, weakly hydrophobic amino acids (Pro, Ala, Gly, Ser, Thr);
xii) Hydrophilic amino acids (Arg, Asn, Asp, Glu, Gln, His, Lys, Ser, Thr, Tyr); and
xiii) Hydrophobic amino acids (Ala, Cys, Gly, Ile, Leu, Met, Phe, Pro, Trp, Val).
xiv) Charged amino acids (Arg, Lys, Asp, Glu)

Since the present polypeptides are utilized in therapeutics which requires the peptides to be in soluble form, the polypeptides of some embodiments of the invention preferably include one or more non-natural or natural polar amino acids, including but not limited to serine, which are capable of increasing solubility of the polypeptide due to their hydroxyl-containing side chain.

According to a specific embodiment, the amino acid sequence of the GCase variant comprises a mutation e.g., substitution as compared to SEQ ID NO: 2.

According to a specific embodiment, the amino acid sequence of the GCase variant comprises a mutation e.g., substitution as compared to SEQ ID NO: 25.

According to an embodiment the mutation(s) is on SEQ ID NO: 25.

The polypeptide of some embodiments of the present invention may comprise a mutation as described herein, as long as the modified regions are not part of the catalytic domain, e.g. do not modify the 3D structure of the catalytic domain which forms the active site (discussed above), or of the glycosylation sites (discussed above), i.e. of SEQ ID NO: 2 or of SEQ ID NO: 25.

According to one embodiment, the GCase polypeptide comprises 4-80, 4-75, 4-70, 4-60, 4-50, 4-45, 4-40, 4-35, 4-30, 4-25, 4-20, 4-15, 4-10, 4-5, 6-80, 6-75, 6-70, 6-60, 6-50, 6-40, 6-35, 6-30, 6-25, 6-20, 6-15, 6-10, 9-80, 9-75, 9-70, 9-60, 9-50, 9-40, 9-35, 9-30, 9-25, 9-20, 9-15, 9-10, 12-80, 12-75, 12-70, 12-60, 12-50, 12-40, 12-35, 12-30, 12-25, 12-20, 12-15, 16-80, 16-75, 16-70,16-60, 16-50, 16-40, 16-35, 16-30, 16-25, 16-20, 19-80, 19-75, 19-70,19-60, 19-50, 19-40, 19-35, 19-30, 19-25, 19-20, 21-80, 21-75, 21-70, 21-60, 21-50, 21-45, 21-40, 21-35, 21-30, 21-25, 25-80, 25-75, 25-70, 25-60, 25-50, 25-45, 25-40, 25-35, 25-30, 30-80, 30-75, 30-70, 30-60, 30-50, 30-45, 30-40, 30-35, 40-80, 40-70, 40-60, 40-50, 50-80, 50-70, 50-55, 55-60, 60-70 or 70-80 mutations in the amino acid sequence set forth in SEQ ID NO: 2 or in SEQ ID NO: 25 (UniProtKB - P04062 (GLCM_HUMAN).

According to one embodiment, the GCase polypeptide comprises 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79 or 80 mutations in the amino acid sequence set forth in SEQ ID NO: 2 or in SEQ ID NO: 25 (UniProtKB - P04062 (GLCM_HUMAN).

According to a specific embodiment, the GCase polypeptide comprises 4 mutations in the amino acid sequence set forth in SEQ ID NO: 2.

According to a specific embodiment, the GCase polypeptide comprises 9 mutations in the amino acid sequence set forth in SEQ ID NO: 2.

According to a specific embodiment, the GCase polypeptide comprises 16 mutations in the amino acid sequence set forth in SEQ ID NO: 2.

According to a specific embodiment, the GCase polypeptide comprises 15 mutations in the amino acid sequence set forth in SEQ ID NO: 25 (UniProtKB - P04062 (GLCM_HUMAN). According to a specific embodiment, the GCase polypeptide comprises 19 mutations in the amino acid sequence set forth in SEQ ID NO: 2.

According to a specific embodiment, the GCase polypeptide comprises 18 mutations in the amino acid sequence set forth in SEQ ID NO: 25 (UniProtKB - P04062 (GLCM_HUMAN).

According to a specific embodiment, the GCase polypeptide comprises 21 mutations in the amino acid sequence set forth in SEQ ID NO: 2.

According to a specific embodiment, the GCase polypeptide comprises 20 mutations in the amino acid sequence set forth in SEQ ID NO: 25 (UniProtKB - P04062 (GLCM_HUMAN).

According to a specific embodiment, the GCase polypeptide comprises 30 mutations in the amino acid sequence set forth in SEQ ID NO: 2. According to a specific embodiment, the GCase polypeptide comprises 29 mutations in the amino acid sequence set forth in SEQ ID NO: 25 (UniProtKB - P04062 (GLCM_HUMAN).

According to a specific embodiment, the GCase polypeptide comprises 36 mutations in the amino acid sequence set forth in SEQ ID NO: 2.

According to a specific embodiment, the GCase polypeptide comprises 35 mutations in the amino acid sequence set forth in SEQ ID NO: 25 (UniProtKB - P04062 (GLCM_HUMAN).

According to a specific embodiment, the GCase polypeptide comprises 46 mutations in the amino acid sequence set forth in SEQ ID NO: 2. According to a specific embodiment, the GCase polypeptide comprises 45 mutations in the amino acid sequence set forth in SEQ ID NO: 25 (UniProtKB - P04062 (GLCM_HUMAN).

According to a specific embodiment, the GCase polypeptide comprises 56 mutations in the amino acid sequence set forth in SEQ ID NO: 2.

According to a specific embodiment, the GCase polypeptide comprises 55 mutations in the amino acid sequence set forth in SEQ ID NO: 25 (UniProtKB - P04062 (GLCM_HUMAN).

According to a specific embodiment, the GCase polypeptide comprises 73 mutations in the amino acid sequence set forth in SEQ ID NO: 2.

According to a specific embodiment, the GCase polypeptide comprises 72 mutations in the amino acid sequence set forth in SEQ ID NO: 25 (UniProtKB - P04062 (GLCM_HUMAN).

As discussed above, the GCase polypeptide of some embodiments of the invention comprises the mutations L34P, K224N/G, T369E and N370D where the coordinates correspond to SEQ ID NO: 2. Amino acid coordinates can be adapted by the skilled artisan by amino acid sequence alignments that may be done manually, or using specific bioinformatic tools such as FASTA, L-ALIGN and protein Blast.

According to one embodiment, the GCase polypeptide further comprises at least one of the mutations: H145K/R, I204K, E222K, T334F/Y/K or L372N, where the coordinates correspond to SEQ ID NO: 2.

According to one embodiment, the GCase polypeptide further comprises two of the mutations: H145K/R, I204K, E222K, T334F/Y/K or L372N, where the coordinates correspond to SEQ ID NO: 2. Thus, for example, the GCase polypeptide may further comprise the mutations: H145K/R and I204K; H145K/R and E222K; H145K/R and T334F/Y/K; H145K/R and L372N; I204K and E222K; I204K and T334F/Y/K; I204K and L372N; E222K and T334F/Y/K; E222K and L372N; or T334F/Y/K and L372N, where the coordinates correspond to SEQ ID NO: 2.

According to one embodiment, the GCase polypeptide further comprises three of the mutations: H145K/R, I204K, E222K, T334F/Y/K or L372N, where the coordinates correspond to

SEQ ID NO: 2. Thus, for example, the GCase polypeptide may further comprise the mutations: H145K/R, I204K and E222K; H145K/R, I204K and T334F/Y/K; H145K/R, I204K and L372N; H145K/R, E222K and T334F/Y/K; H145K/R, E222K and L372N; H145K/R, T334F/Y/K and L372N; I204K, E222K and T334F/Y/K; I204K, E222K and L372N; I204K, T334F/Y/K and L372N; or E222K, T334F/Y/K and L372N, where the coordinates correspond to SEQ ID NO: 2.

According to one embodiment, the GCase polypeptide further comprises four of the mutations: H145K/R, I204K, E222K, T334F/Y/K or L372N, where the coordinates correspond to SEQ ID NO: 2. Thus, for example, the GCase polypeptide may further comprise the mutations: I204K, E222K, T334F/Y/K and L372N; H145K/R, E222K, T334F/Y/K and L372N; H145K/R, I204K, T334F/Y/K and L372N; H145K/R, I204K, E222K and L372N; or H145K/R, I204K, E222K and T334F/Y/K, where the coordinates correspond to SEQ ID NO: 2.

According to one embodiment, the GCase polypeptide further comprises all of the mutations: H145K/R, I204K, E222K, T334F/Y/K and L372N, where the coordinates correspond to SEQ ID NO: 2.

According to one embodiment, the GCase polypeptide comprises all of the mutations: L34P, H145K/R, I204K, E222K, K224N/G, T334F/Y/K, T369E, N370D and L372N, where the coordinates correspond to SEQ ID NO: 2. According to one embodiment, the GCase polypeptide further comprises at least one of the mutations: N102D/E, L165Q, Q226T, L241I, S242P, K473W or H495R, where the coordinates correspond to SEQ ID NO: 2.

According to a specific embodiment, the H495R modification in SEQ ID NO: 2 reverses the single arginine (R) to histidine (H) modification of SEQ ID NO: 2 (i.e. back to the WT sequence as set forth in SEQ ID NO: 25).

According to one embodiment, the GCase polypeptide further comprises two of the mutations: N102D/E, L165Q, Q226T, L241I, S242P, K473W or H495R, where the coordinates correspond to SEQ ID NO: 2. Thus, for example, the GCase polypeptide may further comprise the mutations: N102D/E and L165Q; N102D/E and Q226T; N102D/E and L241I; N102D/E and S242P ; N102D/E and K473W; N102D/E and H495R; L165Q and Q226T; L165Q and L241I; L165Q and S242P; L165Q and K473W; L165Q and H495R; Q226T and L241I; Q226T and S242P; Q226T and K473W; Q226T and H495R; L241I and S242P; L241I and K473W; L241I and H495R; S242P and K473W; S242P and H495R; or K473W and H495R, where the coordinates correspond to SEQ ID NO: 2.

According to one embodiment, the GCase polypeptide further comprises three of the mutations: N102D/E, L165Q, Q226T, L241I, S242P, K473W or H495R, where the coordinates correspond to SEQ ID NO: 2. Thus, for example, the GCase polypeptide may further comprise the mutations: N102D/E, L165Q and Q226T; N102D/E, L165Q and L241I; N102D/E, L165Q and S242P; N102D/E, L165Q and K473W; N102D/E, L165Q and H495R; N102D/E, Q226T and L241I; N102D/E, Q226T and S242P; N102D/E, Q226T and K473W; N102D/E, Q226T and H495R; N102D/E, L241I and S242P; N102D/E, L241I and K473W; N102D/E, L241I and H495R; N102D/E,

S242P and K473W; N102D/E, S242P and H495R; N102D/E, K473W and H495R; L165Q, Q226T and L241I; L165Q, Q226T and S242P; L165Q, Q226T and K473W; L165Q, Q226T and H495R; L165Q, L241I and S242P; L165Q, L241I and K473W; L165Q, L241I and H495R; L165Q, S242P and K473W; L165Q, S242P and H495R; L165Q, K473W and H495R; Q226T, L241I and S242P; Q226T, L241I and K473W; Q226T, L241I and H495R; Q226T, S242P and K473W; Q226T, S242P and H495R; L241I, S242P and K473W; L241I, S242P and H495R; or S242P, K473W and H495R, where the coordinates correspond to SEQ ID NO: 2.

According to one embodiment, the GCase polypeptide further comprises four of the mutations: N102D/E, L165Q, Q226T, L241I, S242P, K473W or H495R, where the coordinates correspond to SEQ ID NO: 2. Thus, for example, the GCase polypeptide may further comprise the mutations: N102D/E, L165Q, Q226T and L241I; N102D/E, L165Q, Q226T and S242P; N102D/E, L165Q, Q226T and K473W; N102D/E, L165Q, Q226T and H495R; N102D/E, Q226T, L241I and S242P; N102D/E, Q226T, L241I and K473W; N102D/E, Q226T, L241I and H495R; N102D/E, L241I, S242P and K473W; N102D/E, L241I, S242P and H495R; N102D/E, S242P, K473W and H495R; L165Q, Q226T, L241I and S242P; L165Q, Q226T, L241I and K473W; L165Q, Q226T, L241I and H495R; L165Q, L241I, S242P and K473W; L165Q, L241I, S242P and H495R; L165Q, S242P, K473W and H495R; Q226T, L241I, S242P and K473W; Q226T, L241I, S242P and H495R; or L241I, S242P, K473W and H495R, where the coordinates correspond to SEQ ID NO: 2.

According to one embodiment, the GCase polypeptide further comprises five of the mutations: N102D/E, L165Q, Q226T, L241I, S242P, K473W or H495R, where the coordinates correspond to SEQ ID NO: 2. Thus, for example, the GCase polypeptide may further comprise the mutations: N102D/E, Q226T, L241I, S242P and K473W; N102D/E, Q226T, L241I, S242P and H495R; N102D/E, Q226T, L241I, K473W and H495R; N102D/E, Q226T, S242P, K473W and H495R; N102D/E, L241I, S242P, K473W and H495R; N102D/E, L165Q, S242P, K473W and H495R; N102D/E, L165Q, L241I, K473W and H495R; N102D/E, L165Q, L241I, S242P and H495R; N102D/E, L165Q, L241I, S242P and K473W; N102D/E, L165Q, Q226T, K473W and H495R; N102D/E, L165Q, Q226T, S242P and H495R; N102D, L165Q, Q226T, S242P and K473W; N102D, L165Q, Q226T, L241I and H495R; N102D/E, L165Q, Q226T, L241I and K473W; Q226T, L241I, S242P, K473W and H495R; L165Q, L241I, S242P, K473W and H495R; L165Q, Q226T, S242P, K473W and H495R; L165Q, Q226T, L241I, K473W and H495R; L165Q, Q226T, L241I, S242P and H495R; or L165Q, Q226T, L241I, S242P and K473W, where the coordinates correspond to SEQ ID

According to one embodiment, the GCase polypeptide further comprises six of the mutations:

N102D/E, L165Q, Q226T, L241I, S242P, K473W or H495R, where the coordinates correspond to SEQ ID NO: 2. Thus, for example, the GCase polypeptide may further comprise the mutations: N102D/E, Q226T, L241I, S242P, K473W and H495R; N102D/E, L165Q, L241I, S242P, K473W and H495R; N102D/E, L165Q, Q226T, S242P, K473W and H495R; N102D/E, L165Q, Q226T, L241I, K473W and H495R; N102D/E, L165Q, Q226T, L241I, S242P and H495R; N102D/E, L165Q, Q226T, L241I, S242P and K473W; or L165Q, Q226T, L241I, S242P, K473W and H495R, where the coordinates correspond to SEQ ID NO: 2.

According to one embodiment, the GCase polypeptide further comprises all of the mutations: N102D/E, L165Q, Q226T, L241I, S242P, K473W and H495R, where the coordinates correspond to SEQ ID NO: 2.

According to one embodiment, the GCase polypeptide comprises all of the mutations: L34P, N102D/E, H145K/R, L165Q, I204K, E222K, K224N/G, Q226T, L241I, S242P, T334F/Y/K, T369E, N370D, L372N, K473W and H495R where the coordinates correspond to SEQ ID NO: 2.

According to one embodiment, the GCase polypeptide further comprises at least one of the mutations: I130T, A168S or D263N, where the coordinates correspond to SEQ ID NO: 2.

According to one embodiment, the GCase polypeptide further comprises two of the mutations: I130T, A168S or D263N, where the coordinates correspond to SEQ ID NO: 2. Thus, for example, the GCase polypeptide may further comprise the mutations: I130T and A168S; I130T and D263N; or A168S and D263N, where the coordinates correspond to SEQ ID NO: 2.

According to one embodiment, the GCase polypeptide further comprises all of the mutations: I130T, A168S and D263N, where the coordinates correspond to SEQ ID NO: 2.

According to one embodiment, the GCase polypeptide comprises all of the mutations: L34P, N102D/E, I130T, H145K/R, L165Q, A168S, I204K, E222K, K224N/G, Q226T, L241I, S242P, D263N, T334F/Y/K, T369E, N370D, L372N, K473W and H495R where the coordinates correspond to SEQ ID NO: 2.

According to one embodiment, the GCase polypeptide further comprises at least one of the mutations: R211N or K303R, where the coordinates correspond to SEQ ID NO: 2.

According to one embodiment, the GCase polypeptide further comprises both of the mutations: R211N and K303R, where the coordinates correspond to SEQ ID NO: 2.

According to one embodiment, the GCase polypeptide comprises all of the mutations: L34P, N102D/E, I130T, H145K/R, L165Q, A168S, I204K, R211N, E222K, K224N/G, Q226T, L241I, S242P, D263N, K303R, T334F/Y/K, T369E, N370D, L372N, K473W and H495R where the coordinates correspond to SEQ ID NO: 2.

According to one embodiment, the GCase polypeptide further comprises at least one of the mutations: H60W L103N/E/R, Q166A, H274R, N333D, N386D, R395K, I406T/A or L420M/I, where the coordinates correspond to SEQ ID NO: 2.

According to one embodiment, the GCase polypeptide further comprises two of the mutations: H60W L103N/E/R, Q166A, H274R, N333D, N386D, R395K, I406T/A or L420M/I, where the coordinates correspond to SEQ ID NO: 2. Thus, for example, the GCase polypeptide may further comprise the mutations: H6OW and L103N/E/R; H6OW and Q166A; H6OW and H274R; H6OW and N333D; H6OW and N386D; H6OW and R395K; H6OW and I406T/A; H6OW and L420M/I; L103N/E/R and Q166A; L103N/E/R and H274R; L103N/E/R and N333D; L103N/E/R and N386D; L103N/E/R and R395K; L103N/E/R and I406T/A; L103N/E/R and L420M/I; Q166A and H274R; Q166A and N333D; Q166A and N386D; Q166A and R395K; Q166A and I406T/A; Q166A and L420M/I; H274R and N333D; H274R and N386D; H274R and R395K; H274R and I406T/A; H274R and L420M/I; N333D and N386D; N333D and R395K; N333D and I406T/A; N333D and L420M/I; N386D and R395K; N386D and I406T/A; N386D and L420M/I; R395K and I406T/A; R395K and L420M/I; or I406T/A and L420M/I, where the coordinates correspond to SEQ ID NO: 2.

According to one embodiment, the GCase polypeptide further comprises three of the mutations: H60W L103N/E/R, Q166A, H274R, N333D, N386D, R395K, I406T/A or L420M/I, where the coordinates correspond to SEQ ID NO: 2. Thus, for example, the GCase polypeptide may further comprise the mutations: H60W L103N/E/R and Q166A; H60W L103N/E/R and H274R; H60W L103N/E/R and N333D; H60W L103N/E/R and N386D; H60W L103N/E/R and R395K; H60W L103N/E/R and I406T/A; H60W L103N/E/R and L420M/I; H60W Q166A and H274R; H60W Q166A and N333D; H60W Q166A and N386D; H60W Q166A and R395K; H60W Q166A and I406T/A; H60W Q166A and L420M/I; H60W H274R and N333D; H60W H274R and N386D; H60W H274R and R395K; H60W H274R and I406T/A; H60W H274R and L420M/I; H60W N333D and N386D; H60W N333D and R395K; H60W N333D and I406T/A; H60W N333D and L420M/I; H60W N386D and R395K; H60W N386D and I406T/A; H60W N386D and L420M/I; H60W R395K and I406T/A; H60W R395K and L420M/I; H60W I406T/A and L420M/I; L103N/E/R, Q166A and H274R; L103N/E/R, Q166A and N333D; L103N/E/R, Q166A and N386D; L103N/E/R, Q166A and R395K; L103N/E/R, Q166A and I406T/A; L103N/E/R, Q166A and L420M/I; L103N/E/R, H274R and N333D; L103N/E/R, H274R and N386D; L103N/E/R, H274R and R395K; L103N/E/R, H274R and I406T/A; L103N/E/R, H274R and L420M/I; L103N/E/R, N333D and N386D; L103N/E/R, N333D and R395K; L103N/E/R, N333D and I406T/A; L103N/E/R, N333D and L420M/I; L103N/E/R, N386D and R395K; L103N/E/R, N386D and I406T/A; L103N/E/R, N386D and L420M/I; L103N/E/R, R395K and I406T/A; L103N/E/R, R395K and L420M/I; L103N/E/R, I406T/A and L420M/I; Q166A, H274R and N333D; Q166A, H274R and N386D; Q166A, H274R and R395K; Q166A, H274R and I406T/A; Q166A, H274R and L420M/I; Q166A, N333D and N386D; Q166A, N333D and R395K; Q166A, N333D and I406T/A; Q166A, N333D and L420M/I; Q166A, N386D and R395K; Q166A, N386D and I406T/A; Q166A, N386D and L420M/I; Q166A, R395K and I406T/A; Q166A, R395K and L420M/I; H274R, N333D and N386D; H274R, N333D and R395K; H274R, N333D and I406T/A; H274R, N333D and L420M/I; H274R, N386D and R395K; H274R, N386D and I406T/A; H274R, N386D and L420M/I; H274R, R395K and I406T/A; H274R, R395K and L420M/I; H274R, I406T/A and L420M/I; N333D, N386D and R395K; N333D, N386D and I406T/A; N333D, N386D and L420M/I; N333D, R395K and I406T/A; N333D, R395K and L420M/I; N333D, I406T/A and L420M/I; N386D, R395K and I406T/A; N386D, R395K and L420M/I; N386D, I406T/A and L420M/I; or R395K, I406T/A and L420M/I, where the coordinates correspond to SEQ ID NO: 2.

According to one embodiment, the GCase polypeptide further comprises four of the mutations: H60W L103N/E/R, Q166A, H274R, N333D, N386D, R395K, I406T/A or L420M/I, where the coordinates correspond to SEQ ID NO: 2. Thus, for example, the GCase polypeptide may further comprise the mutations: H60W L103N/E/R, Q166A and H274R; H60W L103N/E/R, Q166A and N333D; H60W L103N/E/R, Q166A and N386D; H60W L103N/E/R, Q166A and R395K; H60W L103N/E/R, Q166A and I406T/A; H60W L103N/E/R, Q166A and L420M/I; H60W Q166A, H274R and N333D; H60W Q166A, H274R and N386D; H60W Q166A, H274R and R395K; H60W Q166A, H274R and I406T/A; H60W Q166A, H274R and L420M/I; H60W Q166A, N333D and N386D; H60W Q166A, N333D and R395K; H60W Q166A, N333D and I406T/A; H60W Q166A, N333D and L420M/I; H60W Q166A, N386D and R395K; H60W Q166A, N386D and I406T/A; H60W Q166A, N386D and L420M/I; H60W Q166A, R395K and I406T/A; H60W Q166A, R395K and L420M/I; H60W Q166A, I406T/A and L420M/I; H60W H274R, N333D and N386D; H60W H274R, N333D and R395K; H60W H274R, N333D and I406T/A; H60W H274R, N333D and L420M/I; H60W N333D, N386D and R395K; H60W N333D, N386D and I406T/A; H60W N333D, N386D and L420M/I; H60W N386D, R395K and I406T/A; H60W N386D, R395K and L420M/I; H60W R395K, I406T/A and L420M/I; L103N/E/R, Q166A, H274R and N333D; L103N/E/R, Q166A, H274R and N386D; L103N/E/R, Q166A, H274R and R395K; L103N/E/R, Q166A, H274R and I406T/A; L103N/E/R, Q166A, H274R and L420M/I; L103N/E/R, H274R, N333D and N386D; L103N/E/R, H274R, N333D and R395K; L103N/E/R, H274R, N333D and I406T/A; L103N/E/R, H274R, N333D and L420M/I; L103N/E/R, N333D, N386D and R395K; L103N/E/R, N333D, N386D and I406T/A; L103N/E/R, N333D, N386D and L420M/I; L103N/E/R, N386D, R395K and I406T/A; L103N/E/R, N386D, R395K and L420M/I; L103N/E/R, R395K, I406T/A and L420M/I; or N386D, R395K, I406T/A and L420M/I, where the coordinates correspond to SEQ ID NO: 2.

According to one embodiment, the GCase polypeptide further comprises five of the mutations: H60W L103N/E/R, Q166A, H274R, N333D, N386D, R395K, I406T/A or L420M/I, where the coordinates correspond to SEQ ID NO: 2. Thus, for example, the GCase polypeptide may further comprise the mutations: H60W L103N/E/R, Q166A, H274R and N333D; H60W L103N/E/R, Q166A, H274R and N386D; H60W L103N/E/R, Q166A, H274R and R395K; H60W L103N/E/R, Q166A, H274R and I406T/A; H60W L103N/E/R, Q166A, H274R and L420M/I; H60W Q166A, H274R, N333D and N386D; H60W Q166A, H274R, N333D and R395K; H60W Q166A, H274R, N333D and I406T/A; H60W Q166A, H274R, N333D and L420M/I; H60W H274R, N333D, N386D and R395K; H60W H274R, N333D, N386D and I406T/A; H60W H274R, N333D, N386D and L420M/I; H60W N333D, N386D, R395K and I406T/A; H60W N333D, N386D, R395K and L420M/I; H60W N386D, R395K, I406T/A and L420M/I; L103N/E/R, Q166A, H274R, N333D and N386D; L103N/E/R, Q166A, H274R, N333D and R395K; L103N/E/R, Q166A, H274R, N333D and I406T/A; L103N/E/R, Q166A, H274R, N333D and L420M/I; L103N/E/R, H274R, N333D, N386D and R395K; L103N/E/R, H274R, N333D, N386D and I406T/A; L103N/E/R, H274R, N333D, N386D and L420M/I; L103N/E/R, N333D, N386D, R395K and I406T/A; L103N/E/R, N333D, N386D, R395K and L420M/I; L103N/E/R, N386D, R395K, I406T/A and L420M/I; Q166A, H274R, N333D, N386D and R395K; Q166A, H274R, N333D, N386D and I406T/A; Q166A, H274R, N333D, N386D and L420M/I; Q166A, N333D, N386D, R395K and I406T/A; Q166A, N333D, N386D, R395K and L420M/I; Q166A, N386D, R395K, I406T/A and L420M/I; H274R, N333D, N386D, R395K and I406T/A; H274R, N333D, N386D, R395K and L420M/I; or N333D, N386D, R395K, I406T/A and L420M/I, where the coordinates correspond to SEQ ID NO: 2.

According to one embodiment, the GCase polypeptide further comprises six of the mutations: H60W L103N/E/R, Q166A, H274R, N333D, N386D, R395K, I406T/A or L420M/I, where the coordinates correspond to SEQ ID NO: 2. Thus, for example, the GCase polypeptide may further comprise the mutations: H60W L103N/E/R, Q166A, H274R, N333D and N386D; H60W L103N/E/R, Q166A, H274R, N333D and R395K; H60W L103N/E/R, Q166A, H274R, N333D and I406T/A; H60W L103N/E/R, Q166A, H274R, N333D and L420M/I; H60W Q166A, H274R, N333D, N386D and R395K; H60W Q166A, H274R, N333D, N386D and I406T/A; H60W Q166A, H274R, N333D, N386D and L420M/I; H60W H274R, N333D, N386D, R395K and I406T/A; H60W H274R, N333D, N386D, R395K and L420M/I; H60W N333D, N386D, R395K, I406T/A and L420M/I; L103N/E/R, Q166A, H274R, N333D, N386D and R395K; L103N/E/R, Q166A, H274R, N333D, N386D and I406T/A; L103N/E/R, Q166A, H274R, N333D, N386D and L420M/I; L103N/E/R, H274R, N333D, N386D, R395K and I406T/A; L103N/E/R, H274R, N333D, N386D, R395K and L420M/I; L103N/E/R, N333D, N386D, R395K, I406T/A and L420M/I; Q166A, H274R, N333D, N386D, R395K and I406T/A; Q166A, H274R, N333D, N386D, R395K and L420M/I; or H274R, N333D, N386D, R395K, I406T/A and L420M/I, where the coordinates correspond to SEQ ID NO: 2.

According to one embodiment, the GCase polypeptide further comprises seven of the mutations: H60W L103N/E/R, Q166A, H274R, N333D, N386D, R395K, I406T/A or L420M/I, where the coordinates correspond to SEQ ID NO: 2. Thus, for example, the GCase polypeptide may further comprise the mutations: H60W L103N/E/R, Q166A, H274R, N333D, N386D and R395K; H60W L103N/E/R, Q166A, H274R, N333D, N386D and I406T/A; H60W L103N/E/R, Q166A, H274R, N333D, N386D and L420M/I; H60W Q166A, H274R, N333D, N386D, R395K and I406T/A; H60W Q166A, H274R, N333D, N386D, R395K and L420M/I; H60W H274R, N333D, N386D, R395K, I406T/A and L420M/I; L103N/E/R, Q166A, H274R, N333D, N386D, R395K and I406T/A; L103N/E/R, Q166A, H274R, N333D, N386D, R395K and L420M/I; L103N/E/R, H274R, N333D, N386D, R395K, I406T/A and L420M/I; L103N/E/R, Q166A, N333D, N386D, R395K, I406T/A and L420M/I; L103N/E/R, Q166A, H274R, N386D, R395K, I406T/A and L420M/I; L103N/E/R, Q166A, H274R, N333D, R395K, I406T/A and L420M/I; L103N/E/R, Q166A, H274R, N333D, N386D, I406T/A and L420M/I; L103N/E/R, Q166A, H274R, N333D, N386D, R395K and L420M/I; or Q166A, H274R, N333D, N386D, R395K, I406T/A and L420M/I, where the coordinates correspond to SEQ ID NO: 2.

According to one embodiment, the GCase polypeptide further comprises eight of the mutations: H60W L103N/E/R, Q166A, H274R, N333D, N386D, R395K, I406T/A or L420M/I, where the coordinates correspond to SEQ ID NO: 2. Thus, for example, the GCase polypeptide may further comprise the mutations: L103N/E/R, Q166A, H274R, N333D, N386D, R395K, I406T/A and L420M/I; H60W Q166A, H274R, N333D, N386D, R395K, I406T/A and L420M/I; H60W L103N/E/R, H274R, N333D, N386D, R395K, I406T/A and L420M/I; H60W L103N/E/R, Q166A, N333D, N386D, R395K, I406T/A and L420M/I; H60W L103N/E/R, Q166A, H274R, N386D, R395K, I406T/A and L420M/I; H60W L103N/E/R, Q166A, H274R, N333D, R395K, I406T/A and L420M/I; H60W L103N/E/R, Q166A, H274R, N333D, N386D, I406T/A and L420M/I; H60W L103N/E/R, Q166A, H274R, N333D, N386D, R395K and L420M/I; H60W L103N/E/R, Q166A, H274R, N333D, N386D, R395K and I406T/A, where the coordinates correspond to SEQ ID NO: 2.

According to one embodiment, the GCase polypeptide further comprises all of the mutations: H60W L103N/E/R, Q166A, H274R, N333D, N386D, R395K, I406T/A and L420M/I, where the coordinates correspond to SEQ ID NO: 2.

According to one embodiment, the GCase polypeptide comprises at least 4, 5, 6, 7, 8, 9, 10, 11, 12, 13, 14, 15, 16, 17, 18, 19, 20, 21, 22, 23, 24, 25, 26, 27, 28 or 29 of the mutations: L34P, H60W, N102D, L103N, I130T, H145K/R, L165Q, Q166A, A168S, I204K, R211N, E222K, K224N, Q226T, L241I, S242P, D263N, H274R, K303R, N333D, T334F/Y, T369E, N370D, L372N, N386D, R395K, I406T, L420M, K473W, H495R, where the coordinates correspond to SEQ ID NO: 2.

According to one embodiment, the GCase polypeptide comprises all of the mutations: L34P, H60W, N102D, L103N, I130T, H145K/R, L165Q, Q166A, A168S, I204K, R211N, E222K, K224N, Q226T, L241I, S242P, D263N, H274R, K303R, N333D, T334F/Y, T369E, N370D, L372N, N386D, R395K, I406T, L420M, K473W and H495R, where the coordinates correspond to SEQ ID NO: 2.

According to one embodiment, the GCase polypeptide further comprises at least one of the mutations: V78I, A95K, V191M, A322D, V343T, M361E, S364A, H374W, T410E, H451N or L480I, where the coordinates correspond to SEQ ID NO: 2.

According to one embodiment, the GCase polypeptide further comprises at least 2, 3, 4, 5, 6, 7, 8, 9 or 10 of the mutations: V78I, A95K, V191M, A322D, V343T, M361E, S364A, H374W, T410E, H451N or L480I, where the coordinates correspond to SEQ ID NO: 2.

According to one embodiment, the GCase polypeptide further comprises all of the mutations: V78I, A95K, V191M, A322D, V343T, M361E, S364A, H374W, T410E, H451N and L480I, where the coordinates correspond to SEQ ID NO: 2.

According to one embodiment, the GCase polypeptide comprises at least 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34 or 35 of the mutations: L34P, H60W V78I, A95K, N102E, L103E, I130T, H145K/R, L165Q, Q166A, A168S, V191M, I204K, R211N, E222K, K224G, Q226T, L241I, S242P, D263N, A322D, T334K, V343T, M361E, S364A, T369E, N370D, L372N, H374W, I406A, T410E, L420I, H451N, K473W, L480I and/or H495R, where the coordinates correspond to SEQ ID NO: 2.

According to one embodiment, the GCase polypeptide comprises all of the mutations: L34P, H60W V78I, A95K, N102E, L103E, I130T, H145K/R, L165Q, Q166A, A168S, V191M, I204K, R211N, E222K, K224G, Q226T, L241I, S242P, D263N, A322D, T334K, V343T, M361E, S364A, T369E, N370D, L372N, H374W, I406A, T410E, L420I, H451N, K473W, L480I and H495R, where the coordinates correspond to SEQ ID NO: 2.

According to one embodiment, the GCase polypeptide further comprises at least one of the mutations: H162K, S181A, T297S, M335F, K346H, S431A, S465D or A476D, where the coordinates correspond to SEQ ID NO: 2.

According to one embodiment, the GCase polypeptide further comprises at least 2, 3, 4, 5, 6 or 7 of the mutations: H162K, S181A, T297S, M335F, K346H, S431A, S465D or A476D, where the coordinates correspond to SEQ ID NO: 2.

According to one embodiment, the GCase polypeptide further comprises all of the mutations: H162K, S181A, T297S, M335F, K346H, S431A, S465D and A476D, where the coordinates correspond to SEQ ID NO: 2.

According to one embodiment, the GCase polypeptide comprises at least 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44 or 45 of the mutations: L34P, H60W V781, A95K, N102E, L103E, I130T, H145K/R, H162K, L165Q, Q166A, A168S, S181A, V191M, I204K, R211N, E222K, K224G, Q226T, L241I, S242P, D263N, T297S, A322D, T334K, M335F, V343T, K346H, M361E, S364A, T369E, N370D, L372N, H374W, N386D, R395K, I406A, T410E, L4201, S431A, H451N, S465D, K473W, A476D, L4801 and/or H495R, where the coordinates correspond to SEQ ID NO: 2.

According to one embodiment, the GCase polypeptide comprises all of the mutations: L34P, H60W V781, A95K, N102E, L103E, I130T, H145K/R, H162K, L165Q, Q166A, A168S, S181A, V191M, I204K, R211N, E222K, K224G, Q226T, L241I, S242P, D263N, T297S, A322D, T334K, M335F, V343T, K346H, M361E, S364A, T369E, N370D, L372N, H374W, N386D, R395K, I406A, T410E, L4201, S431A, H451N, S465D, K473W, A476D, L4801 and H495R, where the coordinates correspond to SEQ ID NO: 2.

According to one embodiment, the GCase polypeptide further comprises at least one of the mutations: R47K, L51R, Q70H, L91I, G115E, A124G, D140N/G, S196T, or V437S, where the coordinates correspond to SEQ ID NO: 2.

According to one embodiment, the GCase polypeptide further comprises at least 2, 3, 4, 5, 6, 7 or 8 of the mutations: R47K, L51R, Q70H, L91I, G115E, A124G, D140N/G, S196T or V437S, where the coordinates correspond to SEQ ID NO: 2.

According to one embodiment, the GCase polypeptide further comprises all of the mutations: R47K, L51R, Q70H, L91I, G115E, A124G, D140N/G, S196T and V437S, where the coordinates correspond to SEQ ID NO: 2.

According to one embodiment, the GCase polypeptide comprises at least 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54 or 55 of the mutations: L34P, R47K, L51R, H60W Q70H, V781, L91I, A95K, N102E, L103E, G115E, A124G, I130T, D140N/G, H145K/R, H162K, L165Q, Q166A, A168S, S181A, V191M, S196T, I204K, R211N, E222K, K224G, Q226T, L241I, S242P, D263N, T297S, A322D, N333D, T334K, M335F, V343T, K346H, M361E,

S364A, T369E, N370D, L372N, H374W, N386D, R395K, I406A, T410E, L420M, S431A, V437S,

H451N, S465D, K473W, A476D, L480I and/or H495R, where the coordinates correspond to SEQ ID NO: 2.

According to one embodiment, the GCase polypeptide comprises all of the mutations: L34P, R47K, L51R, H60W Q70H, V78I, L91I, A95K, N102E, L103E, G115E, A124G, I130T, D140N/G, H145K/R, H162K, L165Q, Q166A, A168S, S181A, V191M, S196T, I204K, R211N, E222K, K224G, Q226T, L241I, S242P, D263N, T297S, A322D, N333D, T334K, M335F, V343T, K346H, M361E, S364A, T369E, N370D, L372N, H374W, N386D, R395K, I406A, T410E, L420M, S431A, V437S, H451N, S465D, K473W, A476D, L480I and H495R, where the coordinates correspond to SEQ ID NO: 2.

According to one embodiment, the GCase polypeptide further comprises at least one of the mutations: T36Q, S38A, Q143E, T183A, L185M, T272S, H274K, N275D, L286S, K293Q, E300R, K321E, V376T, K408R, Q440E, M450Q and/or I483V, where the coordinates correspond to SEQ ID NO: 2.

According to one embodiment, the GCase polypeptide further comprises at least 2, 3, 4, 5, 6, 7, 8, 9, 10, 11,12,13, 14, 15 or 16 of the mutations: T36Q, S38A, Q143E, T183A, L185M, T272S, H274K, N275D, L286S, K293Q, E300R, K321E, V376T, K408R, Q440E, M450Q and/or I483V, where the coordinates correspond to SEQ ID NO: 2.

According to one embodiment, the GCase polypeptide further comprises all of the mutations: T36Q, S38A, Q143E, T183A, L185M, T272S, H274K, N275D, L286S, K293Q, E300R, K321E, V376T, K408R, Q440E, M450Q and/or I483V, where the coordinates correspond to SEQ ID NO: 2.

According to one embodiment, the GCase polypeptide comprises at least 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71 or 72 of the mutations: L34P, T36Q, S38A, R47K, L51R, H60W Q70H, V78I, L91I, A95K, N102E, L103R, G115E, A124G, I130T, D140G, Q143E, H145R, H162K, L165Q, Q166A, A168S, S181A, T183A, L185M, V191M, S196T, I204K, R211N, E222K, K224G, Q226T, L241I, S242P, D263N, T272S, H274K, N275D, L286S, K293Q, T297S, E300R, K321E, A322D, N333D, T334K, M335F, V343T, K346H, M361E, S364A, T369E, N370D, L372N, H374W, V376T, N386D, R395K, I406A, K408R, T410E, L420M, S431A, V437S, Q440E, M450Q, H451N, S465D, K473W, A476D, L480I, I483V and H495R, where the coordinates correspond to SEQ ID NO: 2 where the coordinates correspond to SEQ ID NO: 2.

According to one embodiment, the GCase polypeptide comprises all of the mutations: L34P, T36Q, S38A, R47K, L51R, H60W Q70H, V78I, L91I, A95K, N102E, L103R, G115E, A124G, I130T, D140G, Q143E, H145R, H162K, L165Q, Q166A, A168S, S181A, T183A, L185M, V191M, S196T, I204K, R211N, E222K, K224G, Q226T, L241I, S242P, D263N, T272S, H274K, N275D, L286S, K293Q, T297S, E300R, K321E, A322D, N333D, T334K, M335F, V343T, K346H, M361E, S364A, T369E, N370D, L372N, H374W, V376T, N386D, R395K, 1406A, K408R, T410E, L420M, S431A, V437S, Q440E, M450Q, H451N, S465D, K473W, A476D, L480I, I483V and H495R, where the coordinates correspond to SEQ ID NO: 2.

As mentioned above, the amino acids of the catalytic domain of the enzyme are not modified.

According to one embodiment, at least 1, 2, 3, 4, 5, 6, 7, 8, 9, 10, 11 or 12 of the amino acids at coordinates D127, F128, W179, N234, E235, Y244, F246, Q284, Y313, E340, 5345, W381, N396 of the GCase polypeptide, where the coordinates correspond to SEQ ID NO: 2 are not modified.

According to one embodiment, the amino acids at all of the following coordinates D127, F128, W179, N234, E235, Y244, F246, Q284, Y313, E340, S345, W381, N396 of the GCase polypeptide, where the coordinates correspond to SEQ ID NO: 2 are not modified.

According to one embodiment, the GCase polypeptide comprises an amino acid sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% identical to a sequence selected from the group consisting of SEQ ID NO: 4, 6, 8, 10, 12, 14, 18, 20, 22 and 27. Such determinations can be carried out using for example the Standard protein-protein BLAST [blastp] software of the NCBI.

According to a specific embodiment, the genetically modified human GCase comprises an amino acid sequence at least 85% identical to SEQ ID NO: 4, 6, 8, 10, 12, 14, 18, 20, 22 or 27.

According to a specific embodiment, the genetically modified human GCase comprises an amino acid sequence at least 90% identical to SEQ ID NO: 4, 6, 8, 10, 12, 14, 18, 20, 22 or 27.

According to a specific embodiment, the genetically modified human GCase comprises an amino acid sequence at least 95% identical to SEQ ID NO: 4, 6, 8, 10, 12, 14, 18, 20, 22 or 27.

According to a specific embodiment, the genetically modified human GCase comprises an amino acid sequence at least 96% identical to SEQ ID NO: 4, 6, 8, 10, 12, 14, 18, 20, 22 or 27.

According to a specific embodiment, the genetically modified human GCase comprises an amino acid sequence at least 97% identical to SEQ ID NO: 4, 6, 8, 10, 12, 14, 18, 20, 22 or 27.

According to a specific embodiment, the genetically modified human GCase comprises an amino acid sequence at least 98% identical to SEQ ID NO: 4, 6, 8, 10, 12, 14, 18, 20, 22 or 27.

According to a specific embodiment, the genetically modified human GCase comprises an amino acid sequence at least 99% identical to SEQ ID NO: 4, 6, 8, 10, 12, 14, 18, 20, 22 or 27.

According to one embodiment, the GCase polypeptide comprises an amino acid sequence identical to a sequence selected from the group consisting of SEQ ID NO: 4, 6, 8, 10, 12, 14, 18, 20, 22 and 27.

According to one embodiment, the GCase polypeptide comprises an amino acid sequence at least 95% identical to SEQ ID NO: 14.

According to one embodiment, the GCase polypeptide comprises an amino acid sequence as set forth in SEQ ID NO: 14.

According to one embodiment, the GCase polypeptide comprises an amino acid sequence at least 95% identical to SEQ ID NO: 22.

According to one embodiment, the GCase polypeptide comprises an amino acid sequence as set forth in SEQ ID NO: 22.

According to one embodiment, the GCase polypeptide comprises an amino acid sequence at least 95% identical to SEQ ID NO: 27.

According to one embodiment, the GCase polypeptide comprises an amino acid sequence as set forth in SEQ ID NO: 27.

The polypeptides of some embodiments of the invention may be synthesized by any techniques that are known to those skilled in the art of polypeptide synthesis. According to one embodiment, polypeptides of the present invention can be synthesized using recombinant DNA technology.

Recombinant techniques are preferably used to generate the polypeptides of the present invention. Such recombinant techniques are described by Bitter et al., (1987) Methods in Enzymol. 153:516-544, Studier et al. (1990) Methods in Enzymol. 185:60-89, Brisson et al. (1984) Nature 310:511-514, Takamatsu et al. (1987) EMBO J. 6:307-311, Coruzzi et al. (1984) EMBO J. 3:1671-1680 and Brogli et al., (1984) Science 224:838-843, Gurley et al. (1986) Mol. Cell. Biol. 6:559-565 and Weissbach & Weissbach, 1988, Methods for Plant Molecular Biology, Academic Press, N.Y., Section VIII, pp 421-463.

To produce a polypeptide of the present invention using recombinant technology, a polynucleotide encoding a polypeptide of the present invention is ligated into a nucleic acid expression construct, which includes the polynucleotide sequence under the transcriptional control of a cis-regulatory (e.g., promoter) sequence suitable for directing constitutive or inducible transcription in the host cells, as further described herein below.

The present teachings also provide for nucleic acid sequences encoding the genetically modified human GCase polypeptides.

As used herein, the phrase “an isolated polynucleotide” refers to a single or a double stranded nucleic acid sequence which is isolated and provided in the form of an RNA sequence, a complementary polynucleotide sequence (cDNA), a genomic polynucleotide sequence and/or a composite polynucleotide sequences (e.g., a combination of the above).

Exemplary polynucleotide sequences for expressing the polypeptides of the present invention are set forth in SEQ ID NOs: 3, 5, 7, 9, 11, 13, 17, 19, 21, 23 or 26.

Other than containing the necessary elements for the transcription and translation of the inserted coding sequence, the expression construct of the present invention can also include sequences (i.e., tags) engineered to enhance stability, production, purification, yield or reduced toxicity of the expressed polypeptide. Such a fusion protein can be designed so that the fusion protein can be readily isolated by affinity chromatography; e.g., by immobilization on a column specific for the heterologous protein. Where a cleavage site is engineered between the peptide moiety and the heterologous protein, the peptide can be released from the chromatographic column by treatment with an appropriate enzyme or agent that disrupts the cleavage site [e.g., see Booth et al. (1988) Immunol. Lett. 19:65-70; and Gardella et al., (1990) J. Biol. Chem. 265:15854-15859].

A variety of prokaryotic or eukaryotic cells can be used as host-expression systems to express the polypeptide coding sequence.

Prokaryotic cells include, but are not limited to, microorganisms, such as bacteria transformed with a recombinant bacteriophage DNA, plasmid DNA or cosmid DNA expression vector containing the polypeptide coding sequence.

Eukaryotic cells include any cell of a eukaryotic organism, including but not limited to, single-and multi-cellular organisms. Single cell eukaryotic organisms include, but are not limited to, yeasts, protozoans, slime molds and algae. Multi-cellular eukaryotic organisms include, but are not limited to, animals (e.g. mammals, insects, invertebrates, nematodes, birds, fish, reptiles and crustaceans), plants, fungi and algae (e.g. brown algae, red algae, green algae).

According to one embodiment, the cell is a plant cell.

The plant cell may are derived from any plant tissue e.g., fruit, flowers, roots, leaves, embryos, embryonic cell suspension, calli or seedling tissue.

According to one embodiment, the eukaryotic cell is not a cell of a plant.

According to one embodiment, the eukaryotic cell is an animal cell.

According to one embodiment, the eukaryotic cell is a cell of a vertebrate.

According to one embodiment, the eukaryotic cell is a cell of an invertebrate.

According to a specific embodiment, the invertebrate cell is a cell of an insect, a snail, a clam, an octopus, a starfish, a sea-urchin, a jellyfish, and a worm.

According to a specific embodiment, the invertebrate cell is a cell of a crustacean. Exemplary crustaceans include, but are not limited to, shrimps, prawns, crabs, lobsters and crayfishes.

According to a specific embodiment, the invertebrate cell is a cell of a fish. Exemplary fish include, but are not limited to, salmon, tuna, pollock, catfish, cod, haddock, sea bass, tilapia, Arctic char and carp.

Mammalian expression systems can also be used to express the polypeptides of the present invention. Cell systems capable of glycosylation of the GCase polypeptide polypeptides are advantageous.

According to one embodiment, the eukaryotic cell is a mammalian cell.

According to a specific embodiment, the mammalian cell is a cell of a non-human organism, such as, but not limited to, a rodent, a rabbit, a pig, a goat, a ruminant (e.g. cattle, sheep, antelope, deer, and giraffe), a dog, a cat, a horse, and a non-human primate.

According to a specific embodiment, the eukaryotic cell is a cell of a human being.

According to one embodiment, the eukaryotic cell is a primary cell, a cell line, a somatic cell, a germ cell, a stem cell, an embryonic stem cell, an adult stem cell, a hematopoietic stem cell, a mesenchymal stem cell, an induced pluripotent stem cell (iPS), a gamete cell, a zygote cell, a blastocyst cell, an embryo, a fetus and/or a donor cell.

According to a specific embodiment, the cell is a human embryonic cell.

According to a specific embodiment, the cell is a human embryonic kidney cell.

According to a specific embodiment, the cell is a HEK293T cell.

According to a specific embodiment, the cell is capable of protein glycosylation (i.e. of the modified human GCase).

The eukaryotic cells may be transformed with recombinant expression vectors containing the polypeptide coding sequence. For example, yeast transformed with recombinant yeast expression vectors containing the polypeptide coding sequence; plant cell systems infected with recombinant virus expression vectors (e.g., cauliflower mosaic virus, CaMV; tobacco mosaic virus, TMV) or transformed with recombinant plasmid expression vectors, such as Ti plasmid, containing the polypeptide coding sequence.

In any case, transformed cells are cultured under effective conditions, which allow for the expression of high amounts of recombinant polypeptides. Effective culture conditions include, but are not limited to, effective media, bioreactor, temperature, pH, and oxygen conditions, which permit protein production. An effective medium refers to any medium in which a cell is cultured to produce the recombinant polypeptides of the present invention. Such a medium typically includes an aqueous solution having assimilable carbon, nitrogen and phosphate sources, and appropriate salts, minerals, metals and other nutrients, such as vitamins. Cells of the present invention can be cultured in conventional fermentation bioreactors, shaker flasks, test tubes, microtiter dishes, and petri plates.

Culturing can be carried out at a temperature, pH and oxygen content appropriate for a recombinant cell. Such culturing conditions are within the expertise of one of ordinary skill in the art.

Depending on the vector and host system used for production, resultant proteins of the present invention may either remain within the recombinant cell; be secreted into the fermentation medium; be secreted into a space between two cellular membranes, such as the periplasmic space in E. coli; or be retained on the outer surface of a cell or viral membrane.

The GCase polypeptide of some embodiments of the invention is endowed with higher expression level and/or higher secretion level from cells (e.g. host expression systems as discussed above) as compared to wild-type human GCase.

The term “wild-type” refers to human glucosylceramidase (human GCase) e.g. as set forth in SEQ ID NO: 2 or SEQ ID NO: 25.

According to one embodiment, the higher expression level and/or higher secretion level is by about 5-25%, 10-50%, 10-100%, 20-90%, 25-75%, 30-80%, 40-50%, 50-60%, 60-70%, 70-80% 90-99% or 95-100%, as compared to wild-type human GCase (e.g. under the same culture conditions).

According to one embodiment, the higher expression level and/or higher secretion level is by about 5%, 10%, 20%, 30%, 40%, 50%, 60%, 70%, 80%, 90%, 95%, 99% or more, as compared to wild-type human GCase (e.g. under the same culture conditions).

According to one embodiment, the GCase polypeptide comprises about 1.3-5 times (e.g. 3 times) higher intracellular expression level in eukaryotic cells as compared to a wild-type human GCase under the same culture conditions (e.g. from HEK293T cells), as discussed below.

According to one embodiment, the GCase polypeptide is secreted from eukaryotic cells (e.g. from HEK293T cells), as compared to a wild-type GCase not being secreted under the same culture conditions, as discussed below.

Methods of assessing expression levels are discussed below.

Following a certain time in culture, recovery of the recombinant protein is effected. The phrase “recovering the recombinant protein” refers to collecting the whole fermentation medium containing the protein, and need not imply additional steps of separation or purification. Proteins of the present invention can be purified using a variety of standard protein purification techniques, such as, but not limited to, affinity chromatography, ion exchange chromatography, filtration, electrophoresis, hydrophobic interaction chromatography, gel filtration chromatography, reverse phase chromatography, concanavalin A chromatography, chromatofocusing, and differential solubilization.

Regardless of the methods by which the GCase variant polypeptide is produced, the genetically modified human GCase maintains the catalytic activity of human GCase. Furthermore, the genetically modified human GCase comprises increased thermal stability. The higher expression levels and the thermal stability, along with the sustained catalytic activity of the GCase polypeptide, is advantageous for Enzyme Replacement Therapy (ERT), specifically for the treatment of Gaucher Disease (discussed below).

As discussed in the Examples section below (see Examples 4, 7 and 10, below), GCase variants D7, D15 and D16 comprise comparable enzymatic activity when compared to wild type GCase and to Cerezyme®, respectively (see Tables 2, 4 and 6, below).

According to one embodiment, the GCase polypeptide is capable of catalyzing hydrolysis of GlcCer similarly to a wild-type GCase under the same conditions (i.e. same experimental conditions, e.g. same buffers, temperature, pH, etc.).

According to one embodiment, the GCase polypeptide is capable of catalyzing hydrolysis of GlcCer, e.g. of the artificial substrate p-nitrophenyl-β-D-glucopyranoside (p-NP-Glc), by about 0.1-2.5×10⁶ k_{cat}/Kₘ (M⁻¹min⁻¹), e.g. 0.15-2.0×10⁶ k_{cat}/Kₘ (M⁻¹min⁻¹).

According to one embodiment, the GCase polypeptide is capable of catalyzing hydrolysis of GlcCer, e.g. of the artificial substrate p-nitrophenyl-β-D-glucopyranoside (p-NP-Glc), by at least about 0.1×10⁶k_{cat}/Kₘ (M⁻¹min⁻¹).

According to one embodiment, the GCase polypeptide is capable of catalyzing hydrolysis of GlcCer, e.g. of the artificial substrate p-nitrophenyl-β-D-glucopyranoside (p-NP-Glc), by at least about 0.2×10⁶k_{cat}/Kₘ (M⁻¹min⁻¹).

According to one embodiment, the GCase polypeptide is capable of catalyzing hydrolysis of GlcCer, e.g. of the artificial substrate p-nitrophenyl-β-D-glucopyranoside (p-NP-Glc), by at least about 0.3×10⁶k_{cat}/Kₘ (M⁻¹min⁻¹).

According to one embodiment, the GCase polypeptide is capable of catalyzing hydrolysis of GlcCer, e.g. of the artificial substrate p-nitrophenyl-β-D-glucopyranoside (p-NP-Glc), by at least about 0.5×10⁶k_{cat}/Kₘ (M⁻¹min⁻¹).

According to a specific embodiment, the GCase polypeptide is capable of catalyzing hydrolysis of p-NP-Glc by at least about 1.0×10⁶ k_{cat}/Kₘ (M⁻¹min⁻¹).

According to a specific embodiment, the GCase polypeptide is capable of catalyzing hydrolysis of p-NP-Glc by at least about 1.5×10⁶ k_{cat}/Kₘ (M⁻¹min⁻¹). According to a specific embodiment, the GCase polypeptide is capable of catalyzing hydrolysis of p-NP-Glc by at least about 1.6×10⁶ k_{cat}/Kₘ (M⁻¹min⁻¹).

According to a specific embodiment, the GCase polypeptide is capable of catalyzing hydrolysis of p-NP-Glc by at least about 1.7×10⁶ k_{cat}/Kₘ (M⁻¹min-1).

According to a specific embodiment, the GCase polypeptide is capable of catalyzing hydrolysis of p-NP-Glc by at least about 1.8×10⁶ k_{cat}/Kₘ (M⁻¹min⁻¹).

Methods of measuring catalytic efficiency of the GCase polypeptides described herein are known in the art and include, for example, in situ activity assay (using e.g., a substrate applied on the cells containing an active enzyme), in vitro activity assays (in which the activity of a particular enzyme is measured in a protein mixture extracted from the cells). For example, an enzyme activity assay may be performed using p-nitrophenyl-β-D-glucopyranoside (p-NP-Glc) as the substrate [discussed in Wei R.R. et al., J. Biol. Chem. (2011) 286: 299-308, incorporated herein by reference].

As discussed in the Examples section below (see Examples 3, 6 and 9, below), GCase variants D7, D15 and D16 comprise a higher thermal stability as compared to wild-type GCase and to

Cerezyme®. Specifically, at pH 6.1, D7 GCase showed an increase of about 6-7° C. when compared to wild-type GCase and of about 11° C. when compared to Cerezyme® (see Table 1, below). At the same pH level, D15 GCase showed a substantial increase of about 12-13° C. when compared to wild-type GCase and a substantial increase of about 17° C. when compared to ^{Cerezyme}® (see Tables 1 and 3, below). Similarly D16 GCase showed a substantial increase of about 19° C. when compared to

C_{erezyme}® (see Table 5, below).

According to one embodiment, the GCase polypeptide comprises a thermal stability in a temperature range 3-22° C. (e.g. 15-22° C., 10-20° C., 5-15° C., 7-13° C., 9-11° C.) higher compared to a wild-type polypeptide under the same conditions (e.g. at a pH of 6.1). The terms “thermal stability” or “increased thermal stability” relative to the wild-type polypeptide means that the GCase polypeptide comprises increased heat stability, i.e. the ability to resist denaturation with increasing temperature. Standard techniques to quantify thermal stability are known in the art, including but not limited to circular dichroism, differential scanning calorimetry and surface plasmon resonance. Methods of measuring thermal stability of the GCase polypeptides described herein are known in the art and include, for example, enzyme stability assay as discussed in Wei R.R. et al., J. Biol. Chem. (2011) 286: 299-308 (incorporated herein by reference).

According to one embodiment, the GCase polypeptide comprises a thermal stability under a temperature being at least about 3° C., 4° C., 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C. or 25° C. higher compared to a wild-type polypeptide under the same conditions (e.g. at a pH of 6.1).

According to a specific embodiment, the GCase polypeptide comprises a thermal stability under a temperature being about 5° C. higher compared to a wild-type polypeptide under the same conditions (e.g. at a pH of 6.1).

According to a specific embodiment, the GCase polypeptide comprises a thermal stability under a temperature being about 7° C. higher compared to a wild-type polypeptide under the same conditions (e.g. at a pH of 6.1).

According to a specific embodiment, the GCase polypeptide comprises a thermal stability under a temperature being about 9° C. higher compared to a wild-type polypeptide under the same conditions (e.g. at a pH of 6.1).

According to a specific embodiment, the GCase polypeptide comprises a thermal stability under a temperature being about 11° C. higher compared to a wild-type polypeptide under the same conditions (e.g. at a pH of 6.1). According to a specific embodiment, the GCase polypeptide comprises a thermal stability under a temperature being about 13° C. higher compared to a wild-type polypeptide under the same conditions (e.g. at a pH of 6.1).

According to a specific embodiment, the GCase polypeptide comprises a thermal stability under a temperature being about 15° C. higher compared to a wild-type polypeptide under the same conditions (e.g. at a pH of 6.1).

According to a specific embodiment, the GCase polypeptide comprises a thermal stability under a temperature being about 17° C. higher compared to a wild-type polypeptide under the same conditions (e.g. at a pH of 6.1).

According to one embodiment, the GCase polypeptide comprises a thermal stability under a temperature range being 3-22° C. (e.g. 15-22° C., 10-20° C., 5-15° C., 7-13° C., 9-11° C.) higher compared to a Cerezyme® polypeptide under the same conditions (e.g. at a pH of 6.1).

The term “Cerezyme®” also referred to as Imiglucerase is a commercial drug for enzyme replacement therapy.

According to one embodiment, the GCase polypeptide comprises a thermal stability under a temperature being at least about 3° C., 4° C., 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C. or 25° C. higher compared to a Cerezyme® polypeptide under the same conditions (e.g. at a pH of 6.1).

According to a specific embodiment, the GCase polypeptide comprises a thermal stability under a temperature being about 5° C. higher compared to a Cerezyme® polypeptide under the same conditions (e.g. at a pH of 6.1).

According to a specific embodiment, the GCase polypeptide comprises a thermal stability under a temperature being about 7° C. higher compared to a ^{Cerezyme}® polypeptide under the same conditions (e.g. at a pH of 6.1).

According to a specific embodiment, the GCase polypeptide comprises a thermal stability under a temperature being about 9° C. higher compared to a Cerezyme® polypeptide under the same conditions (e.g. at a pH of 6.1).

According to a specific embodiment, the GCase polypeptide comprises a thermal stability under a temperature being about 11° C. higher compared to a Cerezyme® polypeptide under the same conditions (e.g. at a pH of 6.1).

According to a specific embodiment, the GCase polypeptide comprises a thermal stability under a temperature being about 13° C. higher compared to a Cerezyme® polypeptide under the same conditions (e.g. at a pH of 6.1). According to a specific embodiment, the GCase polypeptide comprises a thermal stability under a temperature being about 15° C. higher compared to a Cerezyme® polypeptide under the same conditions (e.g. at a pH of 6.1).

According to a specific embodiment, the GCase polypeptide comprises a thermal stability under a temperature being about 17° C. higher compared to a Cerezyme® polypeptide under the same conditions (e.g. at a pH of 6.1).

According to a specific embodiment, the GCase polypeptide comprises a thermal stability under a temperature being about 19° C. higher compared to a Cerezyme® polypeptide under the same conditions (e.g. at a pH of 6.1).

According to one embodiment, the GCase polypeptide comprises a thermal stability under a temperature range being 15-25° C. (e.g. 17-23° C., 19-23° C.) higher compared to a Cerezyme® polypeptide under the same conditions (e.g. at a pH of 7.4).

According to one embodiment, the GCase polypeptide comprises a thermal stability under a temperature being about 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C. or 30° C., higher compared to a Cerezyme® polypeptide under the same conditions (e.g. at a pH of 7.4).

According to a specific embodiment, the GCase polypeptide comprises a thermal stability under a temperature being about 15° C. higher compared to a Cerezyme® polypeptide under the same conditions (e.g. at a pH of 7.4).

According to a specific embodiment, the GCase polypeptide comprises a thermal stability under a temperature being about 17° C. higher compared to a ^{Cerezyme}® polypeptide under the same conditions (e.g. at a pH of 7.4).

According to a specific embodiment, the GCase polypeptide comprises a thermal stability under a temperature being about 20° C. higher compared to a ^{Cerezyme}® polypeptide under the same conditions (e.g. at a pH of 7.4).

According to a specific embodiment, the GCase polypeptide comprises a thermal stability under a temperature being about 22° C. higher compared to a Cerezyme® polypeptide under the same conditions (e.g. at a pH of 7.4).

According to a specific embodiment, the GCase polypeptide comprises a thermal stability under a temperature being about 25° C. higher compared to a Cerezyme® polypeptide under the same conditions (e.g. at a pH of 7.4).

As mentioned above, the GCase polypeptide is highly expressed and secreted from eukaryotic cells. Expression in eukaryotic cells enables glycosylation of GCase, which is vital for GCase activity.

According to another embodiment, the GCase polypeptide is secreted from eukaryotic cells as compared to a wild-type polypeptide not being secreted under the same culture conditions.

According to one embodiment, the GCase polypeptide is secreted about 1.5-10 times (e.g. about 1.5-2 times, about 2-3 times, about 3-4 times, about 4-5 times, about 5-6 times, about 6-7 times, about 8-9 times, about 9-10 times) higher from eukaryotic cells (e.g. from HEK293T cells), as compared to a wild-type GCase under the same culture conditions. According to one embodiment, the GCase polypeptide is secreted about 3 times higher from eukaryotic cells (e.g. from HEK293T cells), as compared to a wild-type GCase under the same culture conditions.

According to one embodiment, the GCase polypeptide is secreted about 5 times higher from eukaryotic cells (e.g. from HEK293T cells), as compared to a wild-type GCase under the same culture conditions.

According to one embodiment, the GCase polypeptide is secreted about 10 times higher from eukaryotic cells (e.g. from HEK293T cells), as compared to a wild-type GCase under the same culture conditions.

According to one embodiment, the GCase polypeptide comprises about 1.3-5 times (e.g. about 1.3-2 times, about 1.3-3 times, about 2-3 times, about 2-4 times, about 3-4 times, about 4-5 times) higher intracellular expression level in eukaryotic cells as compared to a wild-type human GCase under the same culture conditions (e.g. from HEK293T cells).

According to one embodiment, the GCase polypeptide comprises at least about 1.3 times higher intracellular expression level in eukaryotic cells as compared to a wild-type polypeptide under the same culture conditions.

According to one embodiment, the GCase polypeptide comprises about 2 times higher intracellular expression level in eukaryotic cells as compared to a wild-type human GCase under the same culture conditions (e.g. from HEK293T cells).

According to one embodiment, the GCase polypeptide comprises 3 times higher intracellular expression level in eukaryotic cells as compared to a wild-type human GCase under the same culture conditions (e.g. from HEK293T cells).

According to one embodiment, the GCase polypeptide comprises about 4 times higher intracellular expression level in eukaryotic cells as compared to a wild-type human GCase under the same culture conditions (e.g. from HEK293T cells).

As used herein, the phrases “level of expression” and “expression level” refers to the degree of gene expression and/or gene product activity in a biological sample (e.g. eukaryotic cell).

It should be noted that the level of expression can be determined in arbitrary absolute units, or in normalized units (relative to known expression levels of a control reference).

According to one embodiment, the secretion level of the GCase polypeptide from eukaryotic cells may be higher by at least about 5%, 10%, 15%, 20%, 25%, 30%, 40%, 50%, 60%, 70%, 80%, 90% or 100% as compared to that of the wild-type polypeptide under the same culture conditions.

According to one embodiment, the intracellular expression level of the GCase polypeptide in eukaryotic cells may be higher by at least about 5%, 10%, 15%, 20%, 25%, 30%, 40%, 50%, 60%, 70%, 80%, 90% or 100% as compared to that of the wild-type polypeptide under the same culture conditions.

According to specific embodiments the amount of expression is determined using an RNA and/or a protein detection method.

Non-limiting examples of methods of detecting the level of RNA expressed in cells include Northern Blot analysis, RT-PCR analysis, RNA in situ hybridization stain, and in situ RT-PCR stain.

Non-limiting examples of methods of detecting the level and/or activity of specific protein molecules in a cell include Enzyme linked immunosorbent assay (ELISA), Western blot analysis, immunoprecipitation (IP), radio-immunoassay (RIA), Fluorescence activated cell sorting (FACS), immunohistochemical analysis, in situ activity assay (using e.g., a substrate applied on the cells containing an active enzyme), in vitro activity assays (in which the activity of a particular enzyme is measured in a protein mixture extracted from the cells) and molecular weight-based approach. In case the detection of the expression level of a secreted protein is desired, ELISA assay may be performed on a cell medium of in which the cells have been cultured (i.e. which contains cell-secreted content).

According to one embodiment, there is provided an isolated cell comprising at least one exogenous polynucleotide or construct (as discussed above).

According to one embodiment, the isolated cell is a eukaryotic cell (as discussed above).

The genetically modified human GCase of some embodiments, the isolated polynucleotide of some embodiments, the construct of some embodiments, or the cell of some embodiments of the invention, can be administered to an organism per se, or in a pharmaceutical composition where it is mixed with suitable carriers or excipients.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the genetically modified human GCase accountable for the biological effect.

Hereinafter, the phrases “physiologically acceptable carrier” and “pharmaceutically acceptable carrier”, which may be interchangeably used, refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, intraperitoneal, intranasal, or intraocular injections.

Conventional approaches for drug delivery to the central nervous system (CNS) include: neurosurgical strategies (e.g., intracerebral injection or intracerebroventricular infusion); molecular manipulation of the agent (e.g., production of a chimeric fusion protein that comprises a transport peptide that has an affinity for an endothelial cell surface molecule in combination with an agent that is itself incapable of crossing the BBB) in an attempt to exploit one of the endogenous transport pathways of the BBB; pharmacological strategies designed to increase the lipid solubility of an agent (e.g., conjugation of water-soluble agents to lipid or cholesterol carriers); and the transitory disruption of the integrity of the BBB by hyperosmotic disruption (resulting from the infusion of a mannitol solution into the carotid artery or the use of a biologically active agent such as an angiotensin peptide).

However, each of these strategies has limitations, such as the inherent risks associated with an invasive surgical procedure, a size limitation imposed by a limitation inherent in the endogenous transport systems, potentially undesirable biological side effects associated with the systemic administration of a chimeric molecule comprised of a carrier motif that could be active outside of the CNS, and the possible risk of brain damage within regions of the brain where the BBB is disrupted, which renders it a suboptimal delivery method.

Alternately, one may administer the pharmaceutical composition in a local rather than systemic manner, for example, via injection of the pharmaceutical composition directly into a tissue region of a patient.

Pharmaceutical compositions of some embodiments of the invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with some embodiments of the invention thus may be formulated in conventional manner using one or more physiologically acceptable carriers comprising excipients and auxiliaries, which facilitate processing of the active ingredients into preparations which can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions that can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to some embodiments of the invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances that increase the viscosity of the suspension, such as sodium carboxymethyl cellulose, sorbitol or dextran. Optionally, the suspension may also contain suitable stabilizers or agents that increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of some embodiments of the invention may also be formulated in rectal compositions such as suppositories or retention enemas, using, e.g., conventional suppository bases such as cocoa butter or other glycerides.

Pharmaceutical compositions suitable for use in context of some embodiments of the invention include compositions wherein the active ingredients are contained in an amount effective to achieve the intended purpose. More specifically, a therapeutically effective amount means an amount of active ingredients (genetically modified human GCase) effective to prevent, alleviate or ameliorate symptoms of a disorder (e.g., Gaucher Disease), or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

For example, any in vivo or in vitro assay of GCase activity may be employed such as utilizing the animal models for Gaucher disease discussed in Farfel-Becker et al. [Farfel-Becker, Vitner and Futerman, Dis Model Mech. (2011) 4(6): 746-752].

Toxicity and therapeutic efficacy of the active ingredients described herein can be determined by standard pharmaceutical procedures in vitro, in cell cultures or in experimental animals. The data obtained from these in vitro and cell culture assays and animal studies can be used in formulating a range of dosage for use in humans. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p.1).

Dosage amount and interval may be adjusted individually to provide the active ingredient at a sufficient amount to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions of some embodiments of the invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

It will be appreciated that the kit may further comprise another therapeutic composition for treating Gaucher Disease, e.g. an agent for substrate reduction therapy (SRT).

The genetically modified human GCase of some embodiments of the invention, the polynucleotides encoding same, the expression constructs used for their expression, or the cells of some embodiments of the invention, can be used for treating a disease associated with β-glucocerebrosidase deficiency in a subject in need thereof.

The terms “treating” and “treatment” as used herein refer to abrogating, substantially inhibiting, slowing or reversing the progression of a condition, substantially delaying the appearance of clinical symptoms of a condition, substantially ameliorating clinical symptoms of a condition, or substantially preventing the appearance of clinical symptoms of a condition. The term “treating” as used herein further refers to extending survival or delaying death of patients inflicted with a condition.

As used herein the phrases “subject” and “subject in need thereof” which are interchangeably used herein, refer to a mammal, preferably human beings at any age or gender that suffer from the pathology. This term encompasses individuals who are at risk to develop the pathology. The subject may include e.g. neonatal, infant, juvenile, adolescent, adult and elderly adult.

According to one embodiment, the subject has been diagnosed with a disease associated with the GBA gene.

According to one embodiment, the subject has been diagnosed with a disease associated with reduced β-glucocerebrosidase levels and/or activity.

According to one embodiment, the subject has been diagnosed with a disease associated with β-glucocerebrosidase deficiency.

Exemplary diseases associated with β-glucocerebrosidase deficiency include, but are not limited to, Gaucher Disease, GBA-associated Parkinson's disease, GBA-associated dementia with Lewy bodies, and GBA-associated multiple system atrophy.

According to a specific embodiment, the disease associated with β-glucocerebrosidase deficiency is Gaucher Disease.

The terms “Gaucher's disease”, “Gaucher disease” or “GD” as interchangeably used herein, refer to a lysosomal storage disease (LSD) characterized by accumulation of glucosylceramide (GlcCer, also known as glucocerebroside) in cells, particularly in cells of the mononuclear cell lineage. Glucosylceramide can collect in the spleen, liver, kidneys, lungs, brain and bone marrow. The disease is typically caused by a deficiency of the enzyme glucocerebrosidase (also known as beta-glucosidase, D-glucosyl-N-acylsphingosine glucohydrolase, GCD or GCase; EC 3.2.1.45), a lysosomal enzyme with glucosylceramidase activity that is needed to catalyze the hydrolysis of glucosylceramide/G1cCer.

GD is divided into two major types: neuropathic and non-neuropathic disease, based on the particular symptoms of the disease. In non-neuropathic disease most organs and tissues can be involved, but not the brain. In neuropathic disease (nGD) the brain is also involved.

Type I (or non-neuropathic type, GD1) is the most common form of the disease, occurring in approximately 1 in 50,000 live births. It occurs most often among persons of Ashkenazi Jewish heritage. Symptoms may begin early in life or in adulthood, and include enlarged liver and grossly enlarged spleen (known together as ‘hepatosplenomegaly’); the spleen can rupture and cause additional complications. Spleen enlargement and bone marrow replacement cause anemia, thrombocytopenia and leukopenia. Skeletal weakness and bone disease may be extensive. The brain is not affected pathologically, but there may be lung and, rarely, kidney impairment. Patients in this group usually bruise easily (due to low levels of platelets) and experience fatigue due to low numbers of red blood cells. Depending on disease onset and severity, GD type 1 patients may live well into adulthood. Some patients have a mild form of the disease or may not show any symptoms.

Neuropathic GD (nGD) as used herein encompasses both Type 2 and Type 3 GD.

GD type 2, also referred to as acute infantile neuropathic GD, typically begins within 6 months of birth and has an incidence rate around one 1 in 100,000 live births. Symptoms include an enlarged liver and spleen, extensive and progressive brain damage, eye movement disorders, spasticity, seizures, limb rigidity, and a poor ability to suck and swallow. Affected children usually die by age two.

GD type 3, also referred to as chronic neuropathic GD, can begin at any time in childhood or even in adulthood, and occurs in about one in 100,000 live births. It is characterized by slowly progressive, but milder neurologic symptoms compared to the acute or type 2 version. GD Type 3 has been divided into two variants, termed Types 3b and 3a. Type 3b has earlier onset of massive livers and spleens and the patients can also experience direct involvement of the lungs and rapidly progressive bony disease. Major symptoms include an enlarged spleen and/or liver, seizures, poor coordination, skeletal irregularities, eye movement disorders, blood disorders including anemia, and respiratory problems. Patients often live into their early teen years and adulthood.

According to a specific embodiment, the GD is type 1.

According to one embodiment, the genetically modified human GCase of some embodiments of the invention is used for enzyme replacement therapy.

As used herein “enzyme replacement therapy (ERT)” refers to the exogenous administration of β-glucocerebrosidase (GCase).

According to specific embodiments, the genetically modified human GCase treatment is combined with a substrate reduction therapy agent.

As used herein, the term “Substrate reduction therapy (SRT) agent” refers to an agent (e.g. small molecule) that inhibits the synthesis of the natural substrate of the GCase, i.e. glucosylceramide (or GL1). A number of health regulatory agency-approved versions of SRT are available on the market. Examples include, but are not limited to, Miglustat (Zavesca.RTM.) and Eliglustat Tartrate.

It is expected that during the life of a patent maturing from this application many relevant SRT will be developed and the scope of the term SRT is intended to include all such new technologies a priori.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably, and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

It is understood that any Sequence Identification Number (SEQ ID NO) disclosed in the instant application can refer to either a DNA sequence or an RNA sequence, depending on the context where that SEQ ID NO is mentioned, even if that SEQ ID NO is expressed only in a DNA sequence format or an RNA sequence format. For example, SEQ ID NO: 1 is expressed in a DNA sequence format (e.g., reciting T for thymine), but it can refer to either a DNA sequence that corresponds to an GCase nucleic acid sequence, or the RNA sequence of an RNA molecule nucleic acid sequence. Similarly, though some sequences are expressed in an RNA sequence format (e.g., reciting U for uracil), depending on the actual type of molecule being described, it can refer to either the sequence of a RNA molecule comprising a dsRNA, or the sequence of a DNA molecule that corresponds to the RNA sequence shown. In any event, both DNA and RNA molecules having the sequences disclosed with any substitutes are envisioned.

### EXAMPLES

Reference is now made to the following examples, which together with the above descriptions, illustrate the invention in a non-limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, CA (1990); Marshak et al., “Strategies for Protein Purification and Characterization - A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### General Materials and Experimental Procedures

Materials

Dulbecco's modified Eagle's medium and fetal bovine serum were obtained from Gibco. Penicillin, streptomycin and sodium pyruvate for cell culture were obtained from Biological Industries. Anti-DYKDDDDK G1 affinity resin, DYKDDDDK peptide (SEQ ID NO: 16) and anti-DYKDDDDK tag antibody [HRP] were obtained from GenScript. Nickel beads were obtained from Adar Biotech. Strep-Tactin®XT 4Flow high capacity resign, S trepMAB -Clas sic HRP (anti-Strep) antibody and biotin were purchased from IBA GmbH, Germany. Anti-GCase (C-terminal) antibodies produced in rabbits, Monoclonal Anti-polyHistidine-Peroxidase antibodies produced in mice, polyethyleneimine, defatted bovine serum albumin, protease inhibitor cocktail, deoxyribonuclease, Nonidet™ P 40 Substitute (NP-40) and p-nitrophenyl-β-D-glucopyranoside were all obtained from Sigma-Aldrich. Equipment for SDS-PAGE and Western blotting was supplied by BioRad. Recombinant human glucosylceramidase (WT GCase), expressed in CHO cells, was obtained from R&D Systems, Minn., USA. Imiglucerase (Cerezyme®, Sanofi Genzyme) was obtained as leftovers from treatment of patients.

Generation of a More Stable form of GCase

See Examples Section Below

E. coli Expression and Subsequent Purification of GCase

Wild-type (WT) and four mutant variants of human GCase, whose sequences were generated by use of PROSS, viz., D2, D4, D6, D7, were all expressed in E. coli as pET28-bd-SUMO [Zahradnik J. et al., FEBS J. (2019) 286: 3858-3873] constructs that also contained an N-terminal his-tag for purification. The expressed GCase was isolated from the E. coli lysates using standard Ni²⁺ chelate chromatography, and the bound GCase was released from the column using SUMO protease [Frey S. and Görlich D., J. Chromatogr. A. (2014) 1337: 95-105]. Purity of the protein was assessed on 10% Tris-Glycine SDS-PAGE gels stained with Coomassie blue (Instant blue, Expedeon). GCase was identified by Western blotting, using anti-GCase and anti-his-tag antibodies and by Mass Spectrometry (MS).

Cell Culture and Transfection in Eukaryotic Cells

Wild-type (WT) and the D7 variant DNA sequences of human GCase were cloned into a pCDNA 3.1 (Invitrogen) vector, together with an N-terminal FLAG tag for purification (FIG. 2A). HEK293T cells were cultured in Dulbecco's modified Eagle medium supplemented with 10% fetal bovine serum, 100 IU/ml penicillin, 100 μg/ml streptomycin, and 110 μg/ml sodium pyruvate. The cells were transfected using polyethyleneimine reagent and 10 ᵢ.t.g of plasmid per 10 cm culture dish. Cells and growth medium were collected 36-48 hours after transfection.

GCase Purification

GCase was isolated either from cell pellets or from growth medium using anti-DYKDDDDK affinity resign (FLAG beads) and Strep-Tactin®XT resign (Strep beads). Growth medium was transferred to 250 ml tubes and centrifuged at 10,000 g at 4° C. for 20 minutes. 200 μl of a FLAG beads or Strep beads suspension in 150 mM NaCl/50 mM Tris, pH 7.4, was added to a 50 ml Falcon tube filled with the growth medium, and placed on a rotator at 4° C. overnight to enable binding of the GCase to the beads. Cell pellets were lysed by sonication in the same Tris buffer, containing 1% NP-40, protease inhibitor cocktail (1:500) and deoxyribonuclease (1:200). The lysate was centrifuged at 16,000 g at 4° C. for 20 minutes. The pellet was discarded, and FLAG beads were added to the supernatant (50-150 μl of bead suspension per 1-5 ml of supernatant). The mixture was placed on a rotator for a minimum of 2 hours at 4° C. The beads, either FLAG or Strep beads, were then washed with an excess of the Tris buffer. GCase containing FLAG tag was released by competitive elution in 3 consecutive elution steps, using as the eluting ligand the DYKDDDDK peptide (FLAG peptide, SEQ ID NO: 16) dissolved in sodium citrate buffer (10.4 g trisodium citrate, 3.6 g disodium hydrogen citrate dissolved in 1 1 of double distilled water, 187 mM D-mannitol, and 0.1% (v/v) ml Tween 80, pH was adjusted to 6.1 using citric acid). The protein was further purified and stored in the sodium citrate buffer. The eluted fractions were combined, and underwent size exclusion chromatography (SEC) on an analytical Superdex 200 column. Fractions corresponding to the monomeric peak were collected and concentrated on Amicon Ultracentrifugal filters (10 kDa cut-off, Merck Millipore). The protein concentration was determined from the absorbance at 280 nm, and extinction coefficients were calculated on the basis of amino acid sequence composition (ε_{D7-GCase}=108 290 M⁻¹cm⁻¹; ε_{WT-GCase}=95 800 M⁻¹cm⁻¹). Purity was assessed on 10% Tris-Glycine SDS-PAGE gels stained with Coomassie blue (Instant blue, Expedeon). GCase was identified by Western blotting, using anti-GCase, anti-Strep and anti-DYKDDDDK antibodies, and by mass spectrometry (MS).

Differential Scanning Fluorimetry

Differential scanning fluorimetry (DSF) was performed using a NanoDSF Prometheus NT.48 instrument (NanoTemper, Germany). Samples were heated at 1° C/min steps in the temperature range of 20-95° C. Fluorescence emission of tyrosine and tryptophan was recorded at 330 nm and 350 nm. Data were analyzed using a PR.ThermControl v2.1.1 instrument (NanoTemper, Germany). The melting temperature (Tₘ) was defined as the inflection point of the fluorescence intensity (FI) ratio curve, where R(FI)=FI₃₅₀ₙₘ/FI₃₃₀nm.

Enzymatic Activity Assay Using a Synthetic Substrate (p-NP-Glc)

Specific enzyme activity was determined using p-nitrophenyl-β-D-glucopyranoside (p-NP-Glc) as the substrate. The reaction was stopped by raising the pH to 10, at which pH the p-nitrophenol released is fully ionized, and displays a molar absorption of 20 000 M⁻¹cm⁻¹ at 405 nm. The activity assay was adopted from Wei et al [Wei R.R. et al., J. Biol. Chem. (2011) 286: 299-308]. Briefly, an aliquot of the enzyme was incubated with 0.2-4 mM p-NP-Glc in 0.1% BSA/0.125% sodium taurocholate/0.162% Triton X-100/0.02% sodium azide/0.1 M potassium phosphate, pH 5.9, at 25° C. for 60 minutes. The reaction was stopped by 20-50-fold dilution in 1 M glycine buffer, pH 10, and the absorbance of the p-nitrophenol was measured at 405 nm, in a 1 cm cuvette, using an Agilent Cary 3500 spectrophotometer (Agilent Technologies, USA). Absorbance values were translated into p-nitrophenol concentrations, and Michaelis-Menten plots were constructed and fitted using Origin software (OriginLab). Vₘₐₓ values obtained from the fits were converted to k_{cat} values according to the equation k_{cat}=Vₘₐₓ/c, where c is the molar concentration of the GCase catalytic sites.

Enzymatic Activity Assay Using C₆NBD GlcCer

Variants were tested for enzymatic activity using fluorescently labelled natural substrate of GCase (NBD glucosylceramide (d18:1/6:0) (C6NBD GlcCer)).

Protein preparations were incubated with 20 μM C6-NBD-GlcCer, 20 μM defatted BSA in 50 mM MES buffer pH 5.5 at 37° C. for 5 minutes. Reactions were terminated by addition of 750 μl chloroform/methanol (1:2, v/v), followed by addition of 500 μl of chloroform and 730 μl of double distilled water. Samples were vortexed vigorously and centrifuged for 10 minutes at 2,000 g. The upper phase was aspirated and the lower phase, containing the extracted lipids, was dried under the N2 stream. Lipids were resuspended in chloroform/methanol (9:1, v/v), and separated by TLC using chloroform/methanol/9.8 mM CaCl₂.2H₂O(65/30/8, v/v/v) as the developing solvent. NBD-labeled lipids were visualized using a Typhoon 9410 variable mode imager and quantified by ImageQuantTL

(GE Healthcare, Chalfont St Giles, UK). Activity values were calculated as ᵢimol of substrate turned into product by 1 mg of enzyme in 1 minute (μmol.mg⁻¹.min⁻¹).

### Example 1

### PROSS-Generated Variants

Six GCase variants (designs 2-7, i.e. D2-7, set forth in SEQ ID NOs: 4, 6, 8, 10, 12 and 14, respectively, see FIG. 1A) were designed by use of the PROSS algorithm already used successfully for improving expression levels and stability of several other proteins [see, PCT/IL2016/050812 and Goldenzweig A. et al., Mol. Cell. (2016) 63: 337-346, incorporated herein by reference]. Four of these variants, D2, D4, D6 and D7, were expressed in E. coli and tested for enzymatic activity using a synthetic substrate, p-NP-Glc (data not shown). The sequence of WT GCase is shown in FIG. 1B (SEQ ID NO: 2), and the line below displays the mutations that occur in the D7 variant (SEQ ID NO: 14), which bears the highest number of mutations, i.e. 30. It is also the construct displaying the highest enzymatic activity. For further work, the D7 variant was expressed in HEK293T cells, which are capable of protein glycosylation.

### Example 2

### Expression and Purification of Variant D7 GCase

Both WT hGCase and the D7 variant were expressed in HEK293T cells, and isolated either from cell pellets (intracellular) or from culture medium (secreted). Both WT and D7 GCase were expressed intracellularly, but the D7 variant showed higher expression than the WT. SDS-PAGE of the three eluent fractions obtained from individual preparations, using Coomassie blue staining, is displayed in FIGS. 2B and 2C, GCase was identified as the principal band (marked with arrows), by Western blotting and MS. Only D7 GCase was secreted. A highly purified preparation of the secreted D7 GCase was obtained by a one-step purification using FLAG beads (FIG. 2C).

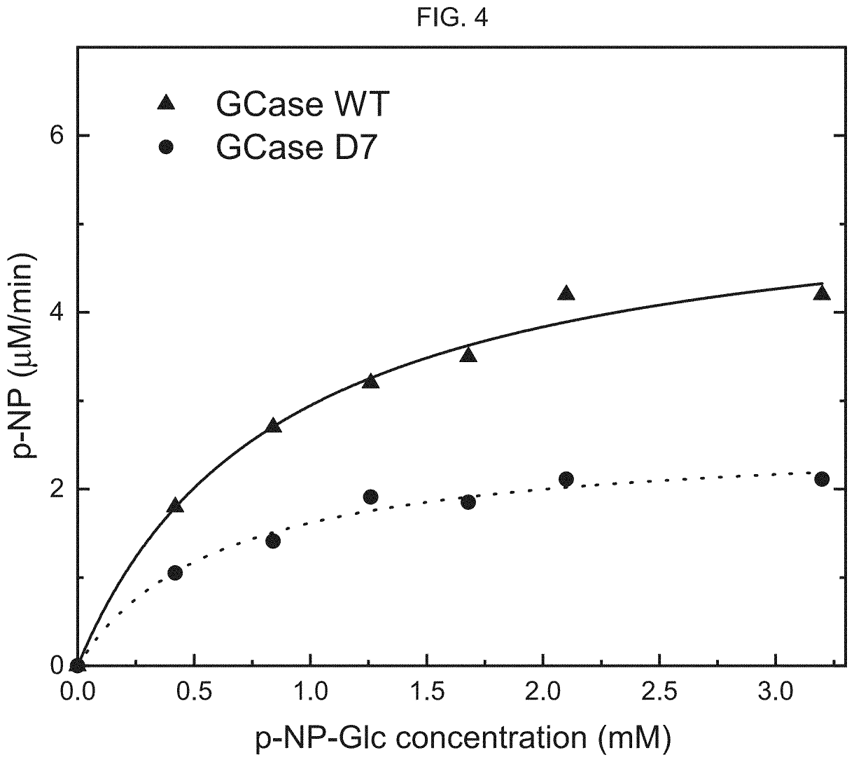

Subsequently, the samples were applied to a Superdex200 column. SEC revealed significant oligomerization of secreted D7 GCase (FIGS. 3A-B). Similar patterns were observed for intracellular WT and D7 GCase. The position of the monomer was established by calibrating the column with molecular weight markers, as the peak with the absorbance maximum at approximately 15 ml (peak 1, FIGS. 3A-B). The monomeric peaks were also shown to be the fractions with the highest specific activity. The fractions corresponding to the monomer were pooled, concentrated, and used for stability and activity assays. In the following, data presented were for the D7 monomer obtained by 2-step purification from the secreted fraction. Due to the extremely low yield of secreted WT GCase, the data obtained for D7 GCase were compared to similar data for recombinant WT

GCase expressed in CHO cells, obtained from R&D Systems, and for Cerezyme®, produced by Sanofi Genzyme, which possesses the WT sequence with a single Arg495His substitution.

### Example 3

### Thermal Stability of Variant D7 GCase

The melting temperature (Tₘ) of D7 GCase was determined using differential scanning fluorimetry (DSF). DSF measures the changes in fluorescence of tyrosine and tryptophan residues upon protein unfolding which results in their exposure to the aqueous environment.

Average Tₘ values for the preparations analyzed are shown in Table 1, below. Tₘ values of 71.8 ±2.4° C. and 61.4 ±1.9° C. for D7 GCase were determined in Tris buffer, pH 7.4, and citrate buffer, pH 6.1, respectively. D7 GCase displayed higher thermal stability than WT GCase. The increase in stability was of about 20° C. and about 11° C., when compared to Cerezyme® at pH 7.4 and pH 6.1, respectively. The values that were obtained for ^{Cerezyme}® and WT GCase using DSF were in good agreement with previously reported Tₘ values obtained for Cerezyme® by differential scanning calorimetry, viz. 51.30 ±0.02° C. at pH 7.1, and 57.67 ±0.04° C. at pH 5.4 [Wei R.R. et al., J. Biol. Chem. (2011) 286: 299-308, supra].

TABLE 1
Tₘ (° C.) measured by differential scanning fluorimetry

| | Tₘ (° C.) pH 7.4 | Tₘ (° C.) pH 6.1 |
|---|---|---|
| GCase WT | — | 55.1 ± 0.5 (n = 2) |
| GCase D7 | 71.8 ± 2.4 (n = 3) | 61.4 ± 1.9 (n > 3) |
| Cerezyme ® | 49.5 ± 1.0 (n > 3) | 50.6 ± 2.2 (n > 3) |

Values are shown for WT GCase, D7 GCase, and Cerezyme ® at two different pH values.

### Example 4

### Specific Activity of variant D7 GCase

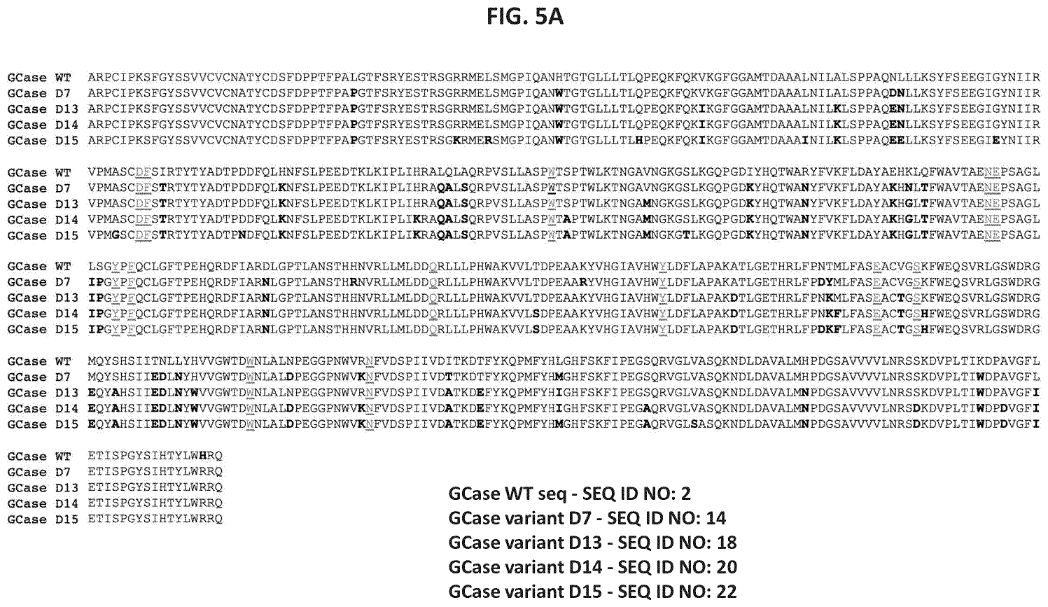

Fitting of the Michaelis-Menten equation to the experimental data permitted to obtain Kₘ and k_{cat} values (Table 2, below, representative enzymatic kinetics data are shown in FIG. 4). The overall catalytic efficiency of the various preparations were compared on the basis of the bimolecular rate constant, k_{cat}/Kₘ. The data obtained showed that the catalytic activity of D7 and the WT does not significantly differ, their k_{cat}/Kₘ values being 0.28×10⁶ and 0.27×10⁶ M⁻¹min⁻¹, respectively. This data thus validated the protocol employed, and the comparative analysis with the artificial substrate, p-nitrophenyl-β-D-glucopyranoside.

TABLE 2
Kinetic parameters

| | | | k_{cat}/Kₘ |
|---|---|---|---|
| | Kₘ (mM) | k_{cat} (min⁻¹) | (M⁻¹min⁻¹) |
| GCase WT | 1.22 ± 0.38 (n = 2) | 347 ± 49 (n = 2) | 0.28 × 10⁶ |
| GCase D7 | 0.89 ± 0.26 (n = 3) | 244 ± 102 (n = 3) | 0.27 × 10⁶ |

Of note, the kinetic parameters were obtained for the activity of GCase preparations on p-nitrophenyl-β-D-glucopyranoside by fitting the Michaelis-Menten curve to the measured data points.

### Example 5

### Additional PROSS-Generated Variants

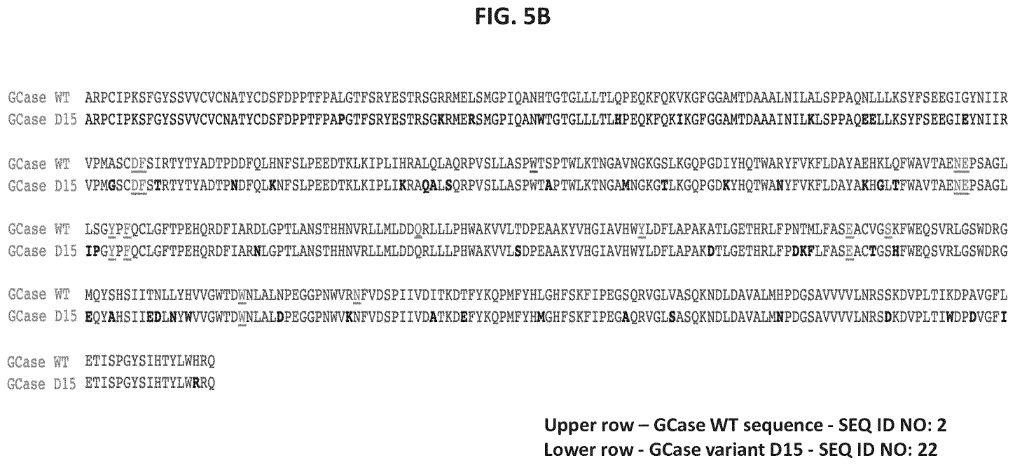

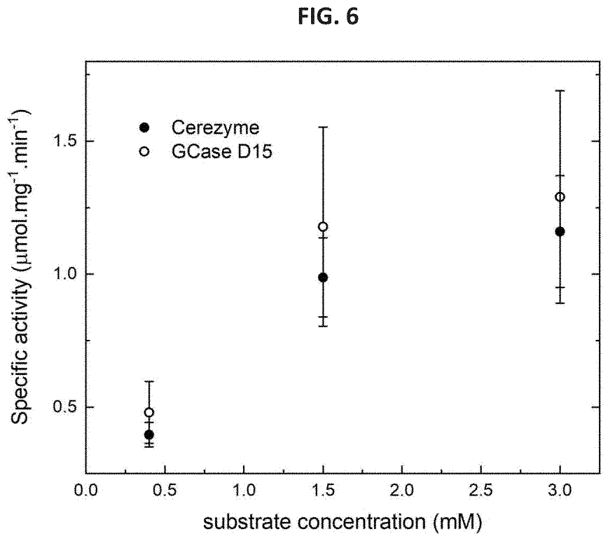

Three additional variants (D13, D14 and D15) of glucosylceramidase (GCase) were designed using the PROSS algorithm previously used successfully for improving expression levels and stability of several other proteins, including GCase (design D7) [see, PCT/IL2016/050812 and Goldenzweig A. et al., (2016) supra, incorporated herein by reference]. GCase variants D13, D14 and D15 were expressed in HEK293T cells and isolated from culture medium. Highly purified preparations of the GCase designs were obtained by a one-step purification using FLAG tag (DYKDDDDK tag, SEQ ID NO: 16) or TwinStrep® tag (SEQ ID NO: 29). All variants were tested for enzymatic activity using fluorescently labelled analogue of GCase (NBD glucosylceramide (d18:1/6:0) (C6NBD GlcCer)) (data not shown). The design with highest enzymatic activity, i.e. variant D15 GCase (as illustrated in FIGS. 5A-B) was used for further characterization. As discussed below, thermal stability and enzymatic activity of the new D15 GCase was compared to previously characterized D7 GCase and to Cerezyme®, produced by Sanofi Genzyme, which possesses the WT sequence with a single Arg495His substitution. All enzymes were kept in 4° C. dissolved in sodium/citrate buffer containing 187 mM D-mannitol, and 0.1% (v/v) Tween 80, pH 6.1.

### Example 6

### Thermal Stability of Variant D15 GCase

The melting temperature (Tₘ) of D15 GCase was determined using differential scanning fluorimetry (DSF). DSF measures the changes in fluorescence of tyrosine and tryptophan residues upon protein unfolding which results in their exposure to the aqueous environment. Experiments were carried out with enzymes dissolved in citrate buffer, pH 6.1 (as discussed above). D7 GCase was shown previously to have approximately 10° C. higher temperature when compared to C_{erezyme}®_{.} Further increase in melting temperature was observed for the variant D15 GCase. The average Tₘ values measured for D15 GCase was 17° C. higher, when compared to ^{Cerezyme}®^{,} reflecting substantial increase in protein thermal stability (Table 3, below). The Tₘ value measured for Cerezyme® was in good agreement with previously reported Tₘ values obtained by differential scanning calorimetry, 51.30 ±0.02° C. at pH 7.1 [Wei R.R. et al., J. Biol. Chem. (2011) 286: 299-308, supra].

TABLE 3
Tₘ (° C.) measured by differential scanning fluorimetry

| | Tₘ (° C.) |
|---|---|
| Cerezyme ® | 50.6 ± 2.2 (n > 3) |
| GCase D7 | 61.4 ± 1.9 (n > 3) |
| GCase D15 | 67.6 ± 1.0 (n = 3) |

### Example 7

### Specific Activity of Variant D15 GCase

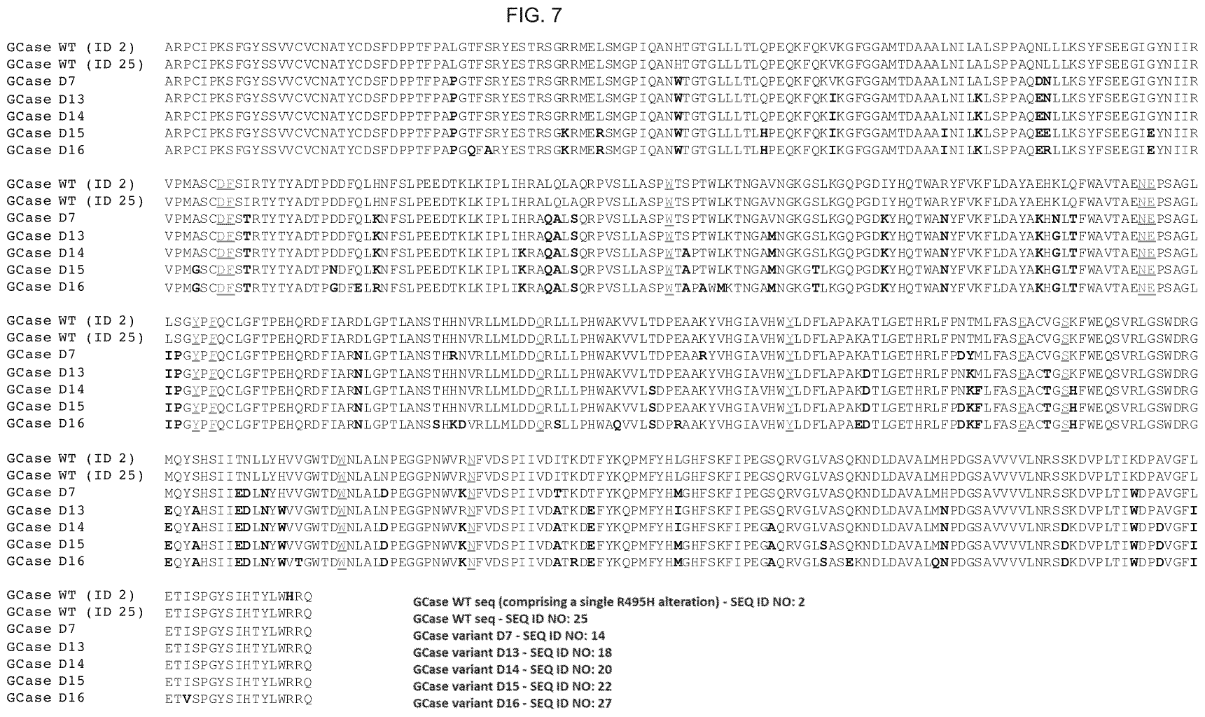

Specific activity was determined by two approaches (i) using fluorescently labelled natural substrate of GCase C6NBD GlcCer (in pH 5.5) and (ii) using artificial substrate p-nitrophenyl-β-D-glucopyranoside (p-NP-Glc) (in pH 5.9). In the first approach, the substrate and product were separated by thin-layer chromatography and quantified by the NBD fluorescence. In the latter, the substrate was quantified spectroscopically, by absorption of created p-nitrophenyl at 405 nm. Specific enzymatic activity determined by both substrates was comparable for commercial Cerezyme® and D15 GCase. Activity values were calculated as μmol of substrate turned into product by 1 mg of enzyme in 1 minute (μmol.mg⁻¹.min⁻¹; Table 4A, below). Substrate concentration was 20 μM for C₆NBD GlcCer assay and 0.4, 1.5 and 3 mM for p-NP-Glc assay (FIG. 6).

TABLE 4A
Specific activity (μmol · mg⁻¹ · min⁻¹)

| | C₆NBD GlcCer | p-NP-Glc (3 mM) |
|---|---|---|
| Cerezyme ® | 0.28 ± 0.12 (n = 3) | 1.16 ± 0.21 (n > 3) |
| GCase D15 | 0.28 ± 0.12 (n > 3) | 1.29 ± 0.41 (n > 3)* |

*Of note, GCase D15 was purified using FLAG tag

A further experiment was carried out to compare the enzymatic kinetics of WT GCase, commercial Cerezyme®, D7 GCase and D15 GCase. The kinetic parameters were obtained for the activity of GCase preparations on p-nitrophenyl-β-D-glucopyranoside (p-NP-Glc) by fitting the Michaelis-Menten curve to the measured data points. The experimental data permitted to obtain Kₘ and k_{cat} values (Table 4B, below). The overall catalytic efficiency of the various preparations were compared on the basis of the bimolecular rate constant, k_{cat}/Kₘ. The data obtained showed that the catalytic activity of D15 was comparable to that of Cerezyme®, their k_{cat}/Kₘ values being 1.49×10⁶ M⁻¹min⁻¹ and 1.53×10⁶ M⁻¹min⁻¹, respectively, while the catalytic activity of D7 was comparable to that of WT GCase, their k_{cat}/Kₘ values being 0.27×10⁶ M⁻¹min⁻¹and 0.28×10⁶M⁻¹min⁻¹, respectively.

TABLE 4B
Kinetic parameters

| | | | k_{cat}/Kₘ |
|---|---|---|---|
| | Kₘ (mM) | k_{cat} (min⁻¹) | (M⁻¹min⁻¹) |
| GCase WT (n = 2) | 1.22 ± 0.38 | 347 ± 49 | 0.28 × 10⁶ |
| Cerezeme ® (n = 3) | 0.7 ± 0.28 | 1071 ± 26 | 1.53 × 10⁶ |
| GCase D7 (n = 3) | 0.89 ± 0.26 | 244 ± 102 | 0.27 × 10⁶ |
| GCase D15 (n = 3) | 0.90 ± 0.23 | 1340 ± 546 | 1.49 × 10⁶ |

### Example 8

### Additional GCase Variant - D16

An additional variant (D16) of glucosylceramidase enzyme (GCase) was designed using the PROSS algorithm previously used successfully for improving expression levels and stability of several other proteins, including GCase (design D7 and D15) [Goldenzweig A. et al., (2016), supra, incorporated herein by reference]. D16 GCase was expressed in HEK 293T cells and isolated from culture medium. A highly purified preparation of the GCase design was obtained by a one-step purification using TwinStrep® tag (SEQ ID NO: 29).

As discussed below, thermal stability and enzymatic activity of the new D16 GCase was compared to previously characterized D15 GCase, purified using TwinStrep® tag (SEQ ID NO: 29), and to Cerezyme®, produced by Sanofi Genzyme, which possesses the WT sequence with a single Arg495His substitution. All enzymes were kept in 4° C. dissolved in sodium/citrate buffer containing 187 mM D-mannitol, and 0.1% (v/v) Tween 80, pH 6.1.

### Example 9

### Thermal Stability of Variant D16 GCase

The melting temperature (Tₘ) of GCase was determined using differential scanning fluorimetry (DSF). DSF measures the changes in fluorescence of tyrosine and tryptophan residues upon protein unfolding which results in their exposure to the aqueous environment.

Experiments were carried out with enzymes dissolved in citrate buffer, pH 6.1 (storage buffer described above). T., values measured previously for GCase D15 were approximately 17° C. higher, when compared to Cerezyme®, reflecting substantial increase in protein thermal stability (Table 3, above, and Table 5, below). The new GCase D16 showed slight increase in Tₘ, namely by 2.5° C. as compared to GCase D15. The Tₘ value measured here for ^{Cerezyme}® was in good agreement with previously reported Tₘ values obtained by differential scanning calorimetry, 51.30 ±0.02° C. at pH 7.1 [Wei R.R. et al., J. Biol. Chem. (2011) 286: 299-308, supra].

TABLE 5
Tₘ (° C.) measured by differential scanning fluorimetry

| | Tₘ (° C.) |
|---|---|
| Cerezyme ® | 50.6 ± 2.2 (n > 3) |
| GCase D7 | 61.4 ± 1.9 (n > 3) |
| GCase D15 | 67.9 ± 0.7 (n > 3) |
| GCase D16 | 70.4 ± 1.0 (n = 3) |

### Example 10

### Specific Activity of Variant D16 GCase

Specific activity was determined using artificial substrate p-nitrophenyl-β-D-glucopyranoside (p-NP-Glc) (in pH 5.9). In this assay the substrate was quantified spectroscopically, by absorption of created p-nitrophenyl at 405 nm. Substrate concentration was 3 mM. Specific enzymatic activity of the new GCase D16 variant was higher than the one determined for commercial Cerezyme® and comparable to GCase D15. Activity values were calculated as μmol of substrate turned into product by 1 mg of enzyme in 1 minute (μmol.mg⁻¹.min⁻¹; Table 6, below).

TABLE 6
Specific activity (μmol · mg⁻¹ · min⁻¹)

| | p-NP-Glc (3 mM)* |
|---|---|
| Cerezyme ® | 1.16 ± 0.21 (n > 3) |
| GCase D15 | 2.67 ± 1.01 (n = 3) |
| GCase D16 | 2.42 ± 0.70 (n = 3) |

*Of note, GCases D15 and D16 were purified using TwinStrep ®

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1. A genetically modified human β-glucocerebrosidase (GCase):
(i) comprising an amino acid sequence at least 85% identical to SEQ ID NO: 2; and
(ii) comprising mutations at coordinates L34P, K224N/G, T369E and N370D, where the coordinates correspond to said SEQ ID NO: 2; and
(iii) capable of catalyzing hydrolysis of a glycolipid glucosylceramide (GlcCer).

2. The genetically modified human GCase of claim 1, further comprising:
(i) at least one of the mutations: H145K/R, I204K, E222K, T334F/Y/K and/or L372N;
(ii) at least one of the mutations: N102D/E, L165Q, Q226T, L241I, S242P, K473W and/or H495R;
(iii) at least one of the mutations: I130T, A168S and/or D263N; (iv) at least one of the mutations: R211N and/or K303R;
(v) at least one of the mutations: H60W L103N/E/R, Q166A, H274R, N333D, N386D, R395K, I406T/A and/or L420M/I;
(vi) at least one of the mutations: V78I, A95K, V191M, A322D, V343T, M361E, S364A, H374W, T410E, H451N and/or L480I;
(vii) at least one of the mutations: H162K, S181A, T297S, M335F, K346H, S431A, S465D and/or A476D;(viii) at least one of the mutations: R47K, L51R, Q70H, L91I, G115E, A124G, D140N/G, S196T and/or V437S; and/or
at least one of the mutations: T36Q, S38A, Q143E, T183A, L185M, T272S, H274K, N275D, L286S, K293Q, E300R, K321E, V376T, K408R, Q440E, M450Q and/or I483V.

3.-10. (canceled)

11. The genetically modified human GCase of claim 1, wherein amino acids at coordinates D127, F128, W179, N234, E235, Y244, F246, Q284, Y313, E340, 5345, W381, N396, where the coordinates correspond to said SEQ ID NO: 2, are not modified.

12. The genetically modified human GCase of claim 1, wherein said amino acid sequence is identical to a sequence selected from the group consisting of SEQ ID NO: 4, 6, 8, 10, 12, 14, 18, 20, 22 and 27.

13. The genetically modified human GCase of claim 12, wherein said amino acid sequence is as set forth in SEQ ID NO: 14.

14. The genetically modified human GCase of claim 12, wherein said amino acid sequence is as set forth in SEQ ID NO: 22.

15. The genetically modified human GCase of claim 12, wherein said amino acid sequence is as set forth in SEQ ID NO: 27.

16. The genetically modified human GCase of claim 1, wherein the genetically modified human GCase is capable of catalyzing hydrolysis of said GlcCer by at least about 0.2×10⁶ k_{cat}/Kₘ (M⁻¹min⁻¹).

17. The genetically modified human GCase of claim 1, wherein the genetically modified human GCase comprises a thermal stability under a temperature range being 5-20° C. higher compared to a wild-type polypeptide under the same conditions.

18. The genetically modified human GCase of any one of claim 1, wherein the genetically modified human GCase comprises at least 2 times higher intracellular expression level in eukaryotic cells as compared to a wild-type polypeptide under the same culture conditions.

19. The genetically modified human GCase of any one of claim 1, wherein the genetically modified human GCase is secreted from eukaryotic cells as compared to a wild-type polypeptide not being secreted under the same culture conditions.

20. An isolated polynucleotide comprising a nucleic acid sequence encoding the genetically modified human GCase of claim 1.

21. The isolated polynucleotide of claim 20, comprising the nucleic acid sequence as set forth in any one of SEQ ID NO: 3, 5, 7, 9, 11, 13, 17, 19, 21, 23 or 26.

22. A nucleic acid construct comprising the isolated polynucleotide of claim 20, and a cis-acting regulatory element for directing expression of said nucleic acid sequence in a cell.

23. The nucleic acid construct of claim 22, wherein said cis-acting regulatory element comprises a promoter.

24. An isolated cell comprising the polynucleotide of claim 20.

25. A pharmaceutical composition comprising as an active ingredient the genetically modified human GCase of claim 1, an isolated polynucleotide encoding same, or a cell comprising same, and a pharmaceutically acceptable carrier or diluent.

26. A method of treating a disease associated with β-glucocerebrosidase deficiency in a subject in need thereof, the method comprising administering to the subject a therapeutically effective amount of the genetically modified human GCase claim 1, an isolated polynucleotide encoding same, or a cell comprising same, thereby treating the disease associated with the β-glucocerebrosidase deficiency in the subject.

27. (canceled)

28. The method of claim 26, wherein the disease associated with β-glucocerebrosidase deficiency is Gaucher disease.

29. The method of claim 26, wherein the subject is a human being.
